(19) United States
(12) Patent Application Publication
(10) Pub. No.: US 2015/0330991 A1
(43) Pub. Date: Nov. 19, 2015

(54) METHOD FOR IMPROVING TARGET AFFINITY OF PEPTIDE

(71) Applicant: GWANGJU INSTITUTE OF SCIENCE AND TECHNOLOGY, Gwangju (KR)

(72) Inventors: Sangyong JON, Daejeon (KR); Sung Hyun KIM, Gwangju (KR); Dae Jin KIM, Gwangju (KR)

(21) Appl. No.: 14/417,666

(22) PCT Filed: Jun. 12, 2013
(86) PCT No.: PCT/KR2013/005196

§ 371 Date: Jul. 20, 2015

(30) Foreign Application Priority Data
Jul. 27, 2012 (KR) 10-2012-0082696

(51) Int. Cl. G01N 33/68; C07K 14/00

(52) U.S. Cl. CPC G01N 33/6845; C07K 14/001

(57) ABSTRACT

The present invention relates to a method for improving the target affinity of a peptide binding to a protein target. The present invention provides a very effective and simple method capable of improving the target affinity of a known peptide in an innovative manner. According to the present invention, the target affinity of the known peptide may be increased, for example, by 1,000 times. The KPI-bipodal peptide binder technique according to the present invention can significantly improve the applicability of the known peptide.

### TECHNICAL FIELD

This application claims priority to and the benefit of Korean Patent Application No. 10-2012-0082696 filed in the Korean Intellectual Property Office on Jul. 27, 2012, the entire contents of which are incorporated herein by reference.

The present invention relates to a method for improving the target affinity of a peptide or bipodal peptide binder (BPB).

### BACKGROUND ART

An antibody is an immunoglobulin protein as a kind of serum protein, which is produced by B cells, and specifically recognizes a particular region of an extraneous antigen to inactivate or incapacitate the antigen. Nowadays, many kinds of antibody products including diagnostic and therapeutic agents have been developed by using the specificity and high affinity of the antigen-antibody reaction and applying the diversity of the antibody capable of discriminating tens of millions of kinds of antigens. Twenty one monoclonal antibodies have been approved by the FDA until now, and antibodies, such as Rituximab and Herceptin, have showed an efficacy in over 50% of patients who exhibit no response to other treatments. Actually, several studies have proved that the use of monoclonal antibodies leads to successful clinic treatment on lymphoma, colorectal cancer, or breast cancer. The whole market size of therapeutic antibodies is supposed to show an average annual growth rate of 20% from 10 billion dollars in 2004 to 30 billion dollars in 2010, and the market size is estimated to grow exponentially. The development of new drug using antibodies is actively made because of a short development period of the drug, a small economical investment cost, and an easy prediction of adverse effects. In addition, the antibody as an herbal medicine has no influence on the human body and has an overwhelmingly longer in vivo half-life than low-molecular weight drugs, and thus the antibody has an affinity to patients. In spite of these availabilities, monoclonal antibodies are recognized as foreign antigens in the human body, causing severe allergic or hypersensitive responses. Furthermore, the clinical use of monoclonal antibodies with anti-cancer activity results in a sharp price rise as a therapeutic agent due to the high production cost thereof and there is an expensive licensing cost since widespread techniques, such as an antibody culturing method and an antibody purification method, are protected by various intellectual property rights.

Therefore, to overcome these drawbacks, the development of antibody alternatives is in the embryonic stage in the EU and especially in the USA. The antibody alternative is a recombinant proteins designed to have constant and variable domains, like an antibody. A predetermined portion of the protein, which is small and stable, is replaced by a random amino acid sequence to produce a library, and the library is used to screen a target material, thereby finding a material with a high affinity and favorable specificity. For example, it has been reported that, of antibody alternatives, avimer and affibody have a picomolar affinity to a target material. It has been reported that these antibody alternatives are small and stable and thus can deeply infiltrate into cancer cells and, in general, induce fewer immune responses. Most of all, these antibody alternatives can avoid a wide range of antibody patent barriers, and have greater economic advantages than antibodies since the antibody alternatives can be easily mass-purified from bacteria, leading to a low production cost. There are 40 antibody alternatives that have been currently been developed, and of these, antibody alternatives which venture companies or multinational pharmaceutical companies attempted to commercialize are fibronectin type III domain, lipocalin, LDLR-A domain, crystalline, protein A, ankyrin repeat, and BPTI, which have high affinity to a target material in the level of picomoles to several nanomoles. Of these, adnectine, avimer, and kunitz domains are currently under FDA clinical trials.

Currently, peptides are variously utilized as alternatives for antibody therapeutic agents since they have appropriate pharmacokinetics, mass productivity, low toxicity, allergenicity suppression, and low production costs as compared with antibodies. The advantages of a peptide as a therapeutic drug include low production costs, high safety and responsiveness, relatively low patent royalty, inhibition of antibody production against the peptide itself due to the rare exposure to the undesirable immune system, and easy and accurate modification through synthesis. However, most of peptides exhibit a low affinity and specificity to a particular protein target compared with antibodies, and thus cannot be used in several application fields. Therefore, it has been urgently demanded in the art to develop novel peptide-based antibody alternatives capable of overcoming drawbacks of peptides.

Hence, the present inventors endeavored to develop a peptide material capable of specifically binding to a biological target molecule in a high affinity, and as a result, have already suggested a bipodal peptide binder (BPB) including a unique structural strategy (see WO 2010/047515). This BPB has a relatively very small molecular weight, but is highly likely to be developed as an antibody alternative due to an excellent affinity to a target.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### DETAILED DESCRIPTION OF THE INVENTION

### Technical Problem

The present inventors have endeavored to develop a method for improving the target affinity of a known target-binding peptide. As a result, the present inventors have established that the target affinity of a known target-binding peptide is greatly increased when a known target-binding peptide has a structure feature of the bipodal peptide binder (BPB) conventionally developed by themselves, and thus have completed the present invention.

Furthermore, the present inventors have endeavored to develop a technique capable of optimizing the target affinity of the bipodal peptide binder conventionally developed by themselves. As a result, the present inventors have developed a method capable of very effectively optimizing or maximizing the target affinity of the BPB while maintaining the overall structure of BPB, and thus have completed the present invention.

Therefore, an aspect of the present invention is to provide a method for increasing the target affinity of a peptide binding to a protein target.

Another aspect of the present invention is to provide a KPI-bipodal peptide binder (BPB) having an increased target affinity.

Therefore, an aspect of the present invention is to provide a method for increasing the target affinity of a bipodal peptide binder (BPB).

Another aspect of the present invention is to provide a bipodal peptide binder (BPB) having an increased target affinity.

Still another aspect of the present invention is to provide a nucleic acid molecule coding a bipodal peptide binder (BPB) having an increased target affinity.

Other purposes and advantages of the present disclosure will become more obvious with the following detailed description of the invention, claims, and drawings.

### Technical Solution

In accordance with an aspect of the present invention, there is provided a method for increasing the target affinity of a peptide binding to a protein target, the method including:

(a) obtaining a known peptide having a binding affinity to the protein target;

(b) providing a library of KPI-bipodal peptide binder including; (i) a structure stabilizing region including parallel, antiparallel, or parallel and antiparallel amino acid strands with interstrand non-covalent bonds; and (ii) a known peptide, as target binding region I, binding to one terminus of the structure stabilizing region, and target binding region II including n amino acids on the other terminus of the structure stabilizing region;

(b) contacting the library with a target; and

(c) selecting a KPI-bipodal peptide binder binding to the target.

In accordance with another aspect of the present invention, there is provided a bipodal peptide binder (BPB) having a target affinity which is increased by the method.

The present inventors have endeavored to develop a method for improving the target affinity of a known target-binding peptide. As a result, the present inventors have established that the target affinity of a known target-binding peptide is greatly increased when a known target-binding peptide has a structure feature of the bipodal peptide binder (BPB) conventionally developed by themselves, the target affinity of the known target-binding peptide is greatly increased.

Herein, the structure of the BPB is basically applied. BPB is a target-affinity peptide that was first proposed by the present inventors, and see WO 2010/047515 for the detailed description of the peptide.

Most of the known peptides have restrictions in several applications due to low target affinities thereof. The present invention suggests a novel and innovative method for improving affinities of the known peptides. The fundamental spirit of the present invention is to provide a target-binding peptide material having an improved target affinity by linking a peptide, which binds to a new binding region adjacent to a region of a target protein to which a known peptide binds, to a structure stabilizing scaffold together with the known peptide.

The method of the present invention improves the affinity of the known peptide, and in this case, the BPB structure of the present inventors is adopted. Therefore, this technique of the present invention is called “KPI-bipodal peptide binder” or “KPI-BPB”.

According to the method of the present invention, a known peptide having a binding affinity to a protein target is first obtained.

As used herein, the term “obtaining a known peptide” refers to synthesizing a known peptide or a nucleic acid sequence coding the known peptide or obtaining information about an amino acid sequence and a nucleic acid sequence, of the known peptide.

The known peptide usable herein includes any known peptide that binds to a protein target.

The protein target herein includes any protein target known in the art. For example, examples of the protein target include enzymes, ligands, receptors, biomarkers, hormones, transcription factors, growth factors, immunoglobulins, signaling proteins, binding proteins, ion channels, antigens, adhesive proteins, structural proteins, regulatory proteins, toxin proteins, cytokines, and blood clotting factors, but are not limited thereto. More specifically, examples of the protein target include fibronectin extra domain B (ED-B), vascular endothelial growth factor (VEGF), vascular endothelial growth factor receptor (VEGFR), vascular cell adhesion molecule-1 (VCAM1), nicotinic acetylcholine receptor (nAchR), human serum albumin (HSA), MyD88, epidermal growth factor receptor (EGFR), HER2/neu, CD20, CD33, CD52, epithelial cell adhesion molecule (EpCAM), immunoglobulin E (IgE), α-chain of lymphocyte function-associated antigen 1 (CD11A), CD3, CD25, glycoprotein IIb/IIIa, integrin, alpha-fetoprotein (AFP), beta₂-microglobulin (β₂M), bladder tumor antigens (BTA), NMP22, cancer antigen 125, cancer antigen 15-3, calcitonin, carcinoembryonic antigen, chromogranin A, estrogen receptor, progesterone receptor, human chorionic gonadotropin (hCG), neuron-specific enolase, prostate-specific antigen (PSA), prostatic acid phosphatase (PAP), thyroglobulin; cytokines [TNF (tumor necrosis factor) alpha, TNF beta, interleukin-10 (IL-10), interferon beta (IFNβ), interferon alpha (IFNα), interferon gamma (IFNγ), granulocyte colony stimulating factor (G-CSF), leukemia inhibitory factor (LIF), human growth hormone (hGH), ciliary neurotrophic factor (CNTF), leptin, oncostatin M, interlukin-6 (IL-6), interlukin-12 (IL-12), EPO(erythropoietin), granulocyte-macrophage colony stimulating factor (GM-CSF), interlukin-2 (IL-2), interlukin-3 (IL-3), interlukin-4 (IL-4), interlukin-5 (IL-5), interlukin-13 (IL-13), Flt13 ligand, and stem cell factor (SCF)]; G-protein coupled receptor (GPCR) [preferably, Class A (or 1) (Rhodopsin-like), Class B (or 2) (Secretin receptor family), Class C (or 3) (Metabotropic glutamate/pheromone), Class D (or 4) (Fungal mating pheromone receptors), Class E (or 5) (Cyclic AMP receptors), and Class F (or 6) (Frizzled/Smoothened) GPCRs, more preferably, a luteinizing hormone receptor, a follicle stimulating hormone receptor, a thyroid stimulating hormone receptor, a calcitonin receptor, a glucagon receptor, a glucagon-like peptide 1 receptor (GLP-1), a metabotropic glutamate receptor, a parathyroid hormone receptor, a vasoactive intestinal peptide receptor, a secretin receptor, a growth hormone releasing factor (GRF) receptor, protease-activated receptor (PARs), cholecystokinin receptor, somatostatin receptor, melanocortin receptor, ADP receptor, adenosine receptor, thromboxane receptor, platelet activating factor receptor, adrenergic receptor, 5-HT receptor, CXCR4, CCR5, chemokine receptor, neuropeptide receptor, opioid receptor, parathyroid hormone (PTH) receptor, ghrelin receptor [symbol] vasoactive intestinal peptide (VIP) receptor, formyl peptide receptor, sex peptide receptor]; receptor tyrosine kinase (RTK) [e.g., epidermal growth factor receptor family, Insulin receptor family, Platelet derived groowth factor receptor family, Fibroblast growth factor receptor family, Vascular endothelial growth factor receptor family, HGF receptor family, Trk receptor family, Eph receptor family, AXL receptor family, LTK receptor family, TIE receptor family, ROR receptor family, DDR receptor family, RET receptor family, KLG receptor family [symbol] RYK receptor family, MuSK receptor family]; and transcription factors [e.g., AP1, AP-2, ARE, Brn-3, C/EBP, CBF, CDP, c-Myb, CREB, E2F-1, EFR, ERE, Ets, Ets-1/PEAS, FAST-1, GAS/ISRE, GATA, GRE, HNF-4, IRF-1, MEF-1, MEF-2, Myc-Max, NF-1, NFATc, NF-E1, NF-E2, NFKB, Oct-1, p53, Pax-5, Pbx1, Pit 1, PPAR, PRE, RAR, RAR (DR-5), SIE, Smad SBE, Smad3/4, SP1, SRE, Stat1, Stat3, Stat4, Stat4, Stat5, Stat6, TFIID, TR, TR(DR-4), USF-1, VDR (DR-3), HSE, and MRE], but are not limited thereto.

The known peptide used herein does not require a particular sequence and length as long as the known peptide has an affinity to a target protein. The known peptide used herein is preferably 4-100 amino acid residues long, more preferably 4-50 amino acid residues long, 5-30 amino acid residues long, 5-20 amino acid residues long, or 5-10 amino acid residues long.

In the KPI-bipodal peptide binder, the structure stabilizing region allows the known peptide, as target binding region I, and target binding region II to bind to a target protein efficiently and stably. Target binding region II is a peptide that binds to a region adjacent to a region of the target protein to which the known peptide binds.

The structure stabilizing region usable herein includes parallel amino acid strands, antiparallel amino acid strands, or parallel and antiparallel amino acid strands, and includes protein structure motifs in which non-covalent bonds are formed by an interstrand hydrogen bond, an electrostatic interaction, a hydrophobic interaction, a Van der Waals interaction, a pi-pi interaction, a cation-pi interaction, or a combination thereof. The non-covalent bonds formed by an interstrand hydrogen bond, an electrostatic interaction, a hydrophobic interaction, a Van der Waals interaction, a pi-pi interaction, a cation-pi interaction, or a combination thereof contribute to the rigidity of the structure stabilizing region.

According to a preferable embodiment of the present invention, the interstrand non-covalent bonds in the structure stabilizing region include a hydrogen bond, a hydrophobic interaction, a Van der Waals interaction, a pi-pi interaction, or a combination thereof.

Optionally, there may be covalent bonds in the structure stabilizing region. For example, the rigidity of the structure stabilizing region can be further enhanced by forming a disulfide bond in the structure stabilizing region. Enhanced rigidity through such covalent bonds is given in consideration of the specificity and affinity of the bipodal peptide binder to a target.

According to a preferable embodiment of the present invention, the amino acid strands of the structure stabilizing region are linked by a linker. As used herein to cite the strand, the term “linker” refers to a material which links between strands. For example, a turn sequence in the β-hairpin serves as a linker in case where the β-hairpin is used as a structure stabilizing region and a material (e.g., peptide linker) that links between two C-termini in a leucine zipper serves as a linker in cases where the leucine zipper is used as a structure stabilizing region.

The linker links parallel, antiparallel, or parallel and antiparallel amino acid strands. For example, at least two strands (preferably, two strands) arranged in a parallel manner, at least two strands (preferably, two strands) arranged in an antiparallel manner, or at least three strands (preferably, three strands) arranged in a parallel and antiparallel manner are linked by a linker.

According to a preferable embodiment of the present invention, the linker is a turn sequence or a peptide linker.

According to a preferable embodiment of the present invention, the turn sequence is a β-turn, γ-turn, α-turn, π-turn, or ω-loop (Venkatachalam C M (1968), Biopolymers, 6, 1425-1436; Nemethy G and Printz M P. (1972), Macromolecules, 5, 755-758; Lewis P N et al., (1973), Biochim. Biophys. Acta, 303, 211-229; Toniolo C (1980) CRC Crit. Rev. Biochem., 9, 1-44; Richardson J S (1981), Adv. Protein Chem., 34, 167-339; Rose G D et al., (1985), Adv. Protein Chem., 37, 1-109; Milner-White E J and Poet R. (1987), TIBS, 12, 189-192; Wilmot C M and Thornton J M (1988), J. Mol. Biol., 203, 221-232; Milner-White E J. (1990), J. Mol. Biol., 216, 385-397; Pavone V et al., (1996), Biopolymers, 38, 705-721; Rajashankar K R and Ramakumar S. (1996), Protein Sci., 5, 932-946). Most preferably, the turn sequence used in the present invention is a β-turn.

In cases where the β-turn is used as a turn sequence, the turn sequence is preferably type I, type I′, type II, type II′, type III, or type III′ turn sequence, more preferably, type I, type I′, type II, or type II′ turn sequence, still more preferably, type I′ or type II′ turn sequence, and most preferably type I′ turn sequence (B. L. Sibanda et al., J. Mol. Biol., 1989, 206, 4, 759-777; B. L. Sibanda et al., Methods Enzymol., 1991, 202, 59-82).

According to another preferable embodiment of the present invention, the sequence usable as a turn sequence in the present invention is disclosed in H. Jane Dyson et al., Eur. J. Biochem. 255:462-471(1998), which is incorporated herein by reference. The sequence usable as a turn sequence includes the following amino acid sequence: X-Pro-Gly-Glu-Val; Ala-X-Gly-Glu-Val (X is selected from 20 amino acids).

According to one embodiment of this invention, it is preferable that, in cases where a β-sheet or leucine zipper is used as a structure stabilizing region, two strands arranged in a parallel manner or two strands arranged in an antiparallel manner are linked by a peptide linker.

As a peptide linker, any one that is known in the art can be used. The sequence of a suitable peptide linker may be selected by considering the following factors: (a) capacity to be applied to a flexible extended conformation; (b) capacity not to form a secondary structure interacting with a biological target molecule; and (c) the absence of a hydrophobic or charged residue interacting with a biological target molecule. A preferable peptide linker includes Gly, Asn, and Ser residues. Other neutral amino acids, such as Thr and Ala, may be included in the linker sequence. The amino acid sequence suitable to the linker is disclosed in Maratea et al., Gene 40:39-46(1985); Murphy et al., Proc. Natl. Acad Sci. USA 83:8258-8562(1986); U.S. Pat. Nos. 4,935,233, 4,751,180, and 5,990,275. The peptide linker sequence may be composed of 1-50 amino acid residues.

According to a preferable embodiment of the present invention, the structure stabilizing region includes a β-hairpin, a β-sheet linked by a linker, or a leucine zipper linked by a linker, more preferably a β-hairpin or a β-sheet linked by a linker, and most preferably, β-hairpin.

As used herein, the term “β-hairpin” refers to the most simple protein motif including two β strands, which are arranged each other in an antiparallel manner. The two β strands in the β-hairpin are generally linked by a turn sequence.

The turn sequence applied to the β-hair pin is preferably type I, type I′, type II, type II′, type III, or type III′ turn sequence, more preferably type I, type I′, type II, type II′ turn sequence, still more preferably type I′ or type II′ turn sequence, and most preferably type I′ turn sequence. In addition, the turn sequence represented by X-Pro-Gly-Glu-Val; or Ala-X-Gly-Glu-Val (X is selected from 20 amino acids) may be also used for the β-hair pin.

According to an exemplary embodiment of the present invention, the type I turn sequence is Asp-Asp-Ala-Thr-Lys-Thr; the type I′ turn sequence is Glu-Asn-Gly-Lys; the type II turn sequence is X-Pro-Gly-Glu-Val or Ala-X-Gly-Glu-Val (X is selected from 20 amino acids), and the type II′ turn sequence is Glu-Gly-Asn-Lys or Glu-D-Pro-Asn-Lys.

Peptides having a β-hairpin conformation are well-known in the art. For example, tryptophan zippers disclosed in U.S. Pat. No. 6,914,123 and Andrea G. Cochran et al., PNAS, 98(10):5578-5583, template-immobilized β-hairpin mimetics disclosed in WO 2005/047503, and β-hairpin modifiers disclosed in U.S. Pat. No. 5,807,979 are well known. Besides, peptides having a β-hair pin confirmation are disclosed in Smith & Regan (1995) Science 270:980-982; Chou & Fassman (1978) Annu. Rev. Biochem. 47:251-276; Kim & Berg (1993) Nature 362:267-270; Minor & Kim (1994) Nature 367:660-663; Minor & Kim (1993) Nature 371:264-267; Smith et al. Biochemistry (1994) 33:5510-5517; Searle et al. (1995) Nat. Struct. Biol. 2:999-1006; Haque & Gellman (1997) J. Am. Chem. Soc. 119:2303-2304; Blanco et al. (1993) J. Am. Chem. Soc. 115:5887-5888; de Alba et al. (1996) Fold. Des. 1: 133-144; de Alba et al. (1997) Protein Sci. 6:2548-2560; Ramirez-Alvarado et al. (1996) Nat. Struct. Biol. 3:604-612; Stanger & Gellman (1998) J. Am. Chem. Soc. 120:4236-4237; Maynard & Searle (1997) Chem. Commun. 1297-1298; Griffiths-Jones et al. (1998) Chem. Commun. 789-790; Maynard et al. (1998) J. Am. Chem. Soc. 120:1996-2007; and Blanco et al. (1994) Nat. Struct. Biol. 1:584-590, which are incorporated herein by reference.

In cases where the peptide having a β-hairpin conformation is used as a structure stabilizing region, a tryptophan zipper is used, most preferably.

According to a preferable embodiment of the present invention, the tryptophan zipper used in the present invention is represented by general formula I below:

X₁-Trp(X₂)X₃-X₄-X₅(X′₂)X₆-X₇ General formula I

wherein X₁ is Ser or Gly-Glu; X₂ and X′₂ each are independently Thr, His, Val, Ile, Phe, or Tyr; X₃ is Trp or Tyr; X₄ is type I, type I′, type II, type II′, type III, or type III′ turn sequence; X₅ is Trp or Phe; X₆ is Trp or Val; and X₇ is Lys or Thr-Glu.

More preferably, in general formula I, X₁ is Ser or Gly-Glu; X₂ and X′₂ each are independently Thr, His, or Val; X₃ is Trp or Tyr; X₄ is type I, type I′, type II, type II′ turn sequence; X₅ is Trp or Phe; X₆ is Trp or Val; and X₇ is Lys or Thr-Glu.

Still more preferably, in general formula I, X₁ is Ser or Gly-Glu; X₂ and X′₂ each are independently Thr, His, or Val; X₃ is Trp; X₄ is type I, type I′, type II, type II′ turn sequence; X₅ is Trp; X₆ is Trp; and X₇ is Lys or Thr-Glu.

Still more preferably, in general formula I, X₁ is Ser; X₂ and X′₂ are Thr; X₃ is Trp; X₄ is type I′ or type II′ turn sequence; X₅ is Trp; X₆ is Trp; and X₇ is Lys.

Most preferably, in general formula I, X₁ is Ser; X₂ and X′₂ are Thr; X₃ is Trp; X₄ is type I′ turn sequence (ENGK) or type II′ turn sequence (EGNK); X₅ is Trp; X₆ is Trp; and X₇ is Lys.

Exemplary amino acid sequences of the tryptophan zipper suitable in the present invention are described in SEQ ID NOs: 1 to 3 and 5 to 10.

The β-hairpin peptide usable as a structure stabilizing region in the present invention is a peptide derived from B1 domain of protein G, i.e. GB1 peptide.

In cases where the GB1 peptide is used, the structure stabilizing region is preferably represented by general formula II below:

X₁-Trp-X₂-Tyr-X₃-Phe-Thr-Val-X₄ General formula II

X₁ is Arg, Gly-Glu, or Lys-Lys; X₂ is Gln or Thr; X₃ is type I, type I′, type II, type II′, type III, or type III′ turn sequence; and X₄ is Gln, Thr-Glu, or Gln-Glu.

More preferably, the structure stabilizing region of general formula II′ is represented by general formula II′ below:

X₁-Trp-Thr-Tyr-X₂-Phe-Thr-Val-X₃ General formula II

X₁ is Gly-Glu or Lys-Lys; X₂ is type I, type I′, type II, type II′, type III, or type III′ turn sequence; and X₃ is Thr-Glu or Gln-Glu.

The exemplary amino acid sequences of the GBA β-hair pin suitable in the present invention are described in SEQ ID NOs: 4, 14, and 15.

The β-hairpin peptide usable as a structure stabilizing region in the present invention is an HP peptide. In cases where the HP peptide is used, the structure stabilizing region is preferably represented by general formula III below:

X₁-X₂-X₃-Trp-X₄-X₅-Thr-X₆-X₇ General formula III

wherein X₁ is Lys or Lys-Lys; X₂ is Trp or Tyr; X₃ is Val or Thr; X₄ is type I, type I′, type II, type II′, type III, or type III′ turn sequence; X₅ is Trp or Ala; X₆ is Trp or Val; and X₇ is Glu or Gln-Glu.

Still another β-hairpin peptide usable as a structure stabilizing region in the present invention is represented by general formula IV below:

X₁-X₂-X₃-Trp-X₄ General formula IV

X₁ is Lys-Thr or Gly; X₂ is Trp or Tyr; X₃ is type I, type I′, type II, type II′, type III, or type III′ turn sequence; and X₄ is Thr-Glu or Gly.

The exemplary amino acid sequences of the β-hair pin of general formulas III and IV are described in SEQ ID NOs: 11, 12, 15, and 16 to 19.

According to the present invention, a β-sheet linked by a linker may be used as a structure stabilizing region. In the β-sheet structure, two strands that are arranged in a parallel or antiparallel manner, preferably in an antiparallel manner, have an extended form, and hydrogen bonds are formed between two strands.

In the β-sheet structure, two adjacent termini of two amino acid strands are linked by a linker. As a linker, the foregoing various turn-sequences or peptide linkers may be used. In cases where the turn-sequence is used as a linker, the β-turn sequence is most preferable.

According to another modification of the present invention, the leucine zipper or the leucine zipper linked by a linker may be used as a structure stabilizing region. The leucine zipper is a conservative peptide domain which causes a dimerization of two parallel α-chains, and a dimerized domain generally found in a protein involved in the gene expression (“Leucine scissors”. Glossary of Biochemistry and Molecular Biology (Revised). (1997). Ed. David M. Glick. London: Portland Press; Landschulz W H, et al. (1988) Science 240:1759-1764). The leucine zipper generally includes a haptad repeat sequence, and a leucine residue is located at the fourth or fifth position. For example, the leucine zipper usable in the present invention includes an amino acid sequence of LEALKEK, LKALEKE, LKKLVGE, LEDKVEE, LENEVAR, or LLSKNYH. A specific example of the leucine zipper used in the present invention is described in SEQ ID NO: 39. The half of each part of the leucine zipper is composed of a short α-chain, and there is a direct leucine contact between the α-chains. The leucine zipper in the transcription factor is generally composed of a hydrophobic leucine zipper region and a basic region (a region interacting with a major groove of the DNA molecule). In cases where the leucine zipper is used in the present invention, the basic region is not necessarily in need. In the leucine zipper structure, two adjacent termini of two amino acid strands (i.e., two α-chains) may be linked by a linker. As a linker, various turn-sequences or peptide linkers as described above may be used. Preferably, a peptide linker that does not influence the structure of the leucine zipper is used.

The known peptide, as target binding region I, and target binding region II, which includes a random amino acid sequence, are linked to both termini of the foregoing structure stabilizing region.

One of the greatest features of the present invention is to manufacture a KPI-bipodal peptide binder by linking a target-binding known peptide to one terminus of the structure stabilizing region and linking the random amino acid sequence to the other terminus of the structure stabilizing region. The known peptide, as target binding region I, and target binding region II, which has a random amino acid sequence, cooperatively bind to the target, thereby greatly increasing an affinity to the target.

The number of amino acids of target binding region II is not particularly limited, and preferably 4-10 amino acid residues, more preferably 4-50 amino acid residues, 5-30 amino acid residues, 5-20 amino acid residues, or 5-10 amino acid residues.

Target binding region I and target binding region II may include the same number or different numbers of amino acid residues.

The amino acid sequence included in target binding region I and/or target binding region II is a linear amino acid sequence or a cyclic amino acid sequence. In order to increase the safety of the peptide sequence of the target biding region, at least one amino acid residue of the amino acid sequence included in target binding region I and/or target binding region II may be modified into an acetyl group, a fluorenyl methoxy carbonyl group, a formyl group, a palmitoyl group, a myristyl group, a stearyl group, or polyethylene glycol (PEG).

The KPI-bipodal peptide binder of the present invention bound to a biological target molecule can be used for the regulation of in vivo physiological response, the detection of in vivo material, in vivo molecule imaging, in vitro cell imaging, and targeting for drug delivery, and may also be used as an escort molecule.

According to a preferable embodiment of the present invention, a functional molecule is further bound to the structure stabilizing region, target binding region I, or target binding region II (more preferably, a structure stabilizing region, and still more preferably, a linker of a structure stabilizing region). Examples of the functional molecule include a label generating a detectable signal, a chemical drug, a bio-drug, a cell penetrating peptide (CPP), and nanoparticles, but are not limited thereto.

The label capable of generating a detectable signal includes T1 contrast materials (e.g., Gd chelate compounds), T2 contrast materials (e.g., superparamagnetic materials (e.g., magnetite, Fe₃O₄, γ-Fe₂O₃, manganese ferrite, cobalt ferrite, and nickel ferrite)), radioactive isotopes (e.g., ¹¹C, ¹⁵O, ¹³N, P³², S³⁵, ⁴⁴Sc, ⁴⁵Ti, ¹¹⁸I, ¹³⁶La, ¹⁹⁸Tl, ²⁰⁰Tl, ²⁰⁵Bi, and ²⁰⁶Bi), fluorescent materials (fluorescein, phycoerythrin, rhodamine, lissamine, and Cy3/Cy5), chemiluminescent materials, magnetic particles, mass labels, and dense electron particles, but are not limited thereto.

Examples of the chemical drug include anti-inflammatory agents, pain relievers, anti-arthritic agents, antispasmodics, anti-depressive agents, antipsychotics, tranquilizers, anti-anxiety drug, narcotic antagonists, anti-Parkinson's disease drugs, cholinergic agonists, anti-cancers, anti-angiogenic agents, immunosuppressive agents, antiviral agents, antibiotics, appetite suppressants, pain relievers, anti-cholinergic agents, anti-histamines, anti-migraine agents, hormonal agents, coronary, cerebral or peripheral vasodilators, contraceptives, anti-thrombotic agents, diuretics, antihypertensive agents, cardiovascular therapeutic agents, and cosmetic ingredients (e.g., anti-wrinkle agents, skin aging inhibitors, and skin whitening agents), but are not limited thereto.

Examples of the biod-rug may include insulin, insulin-like growth factor 1 (IGF-1), growth hormones, erythropoietin, granulocyte-colony stimulating factors (G-CSFs), granulocyte/macrophage-colony stimulating factors (GM-CSFs), interferon-alpha, interferon-beta, interferon-gamma, interlukin-1 alpha and beta, interlukin-3, interlukin-4, interlukin-6, interlukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporine, exedine, lanreotide, luteinizing hormone-releasing hormone (LHRH), nafarelin, parathormone, pramlintide, T-20 (enfuvirtide), thymalfasin, ziconotide, RNA, DNA, cDNA, antisense oligonucleotide, and siRNA, but are not limited thereto.

The KPI-bipodal peptide binder of the present invention may bind to a biomolecule (e.g, protein) exposed on the cell surface, but may also bind to a biomolecule (e.g., protein) in cells to regulate activity of the biomolecule.

In cases where the KPI-bipodal peptide binder targets the protein in cells, the KPI-bipodal peptide binder further includes a cell penetrating peptide (CPP), preferably.

Examples of the CCP include various CCPs known in the art, and include, for example, HIV-1 Tat protein, Tat peptide analogues (e.g., oligo-arginine), ANTP peptide, HSV VP22 transcriptional regulatory protein, MTS peptide derived from vFGF, penetratin, transportan, or Pep-1 peptide, but are not limited thereto. The CPP is bound to the KPI-bipodal peptide by various methods, and for example, the CCP is covalently bound to a lysine residue of a loop region in the structure stabilizing region of the KPI-bipodal peptide.

There are numerous target proteins that play a critical function in the physiological activity in cells, and the CCP-bound KPI-bipodal peptide binder penetrates into cells, and are bound to these target proteins, thereby regulating (e.g., suppressing) activities thereof.

As described above, the KPI-bipodal peptide binder of the present invention has a construct of “known peptide as N-target binding region I—one strand of structure stabilizing region—the other strand of structure stabilizing region—target binding region II-C”.

According to a preferable embodiment of the present invention, the KPI-bipodal peptide binder includes a structure influence inhibiting region which blocks a mutual structural influence between the target binding region and the structure stabilizing region, and th KPI-bipodal peptide binder is placed between target binding region I and one strand of the structure stabilizing region and/or between and the other strand of the structure stabilizing region and target binding region II. Amino acids, of which φ and ψ rotations are relatively free in peptide molecule, are located at the rotation region. Preferably, the amino acids, of which φ and ψ rotations are relatively free, are glycine, alanine, and serine. The number of amino acids that may be located at the structure influence inhibiting region may be 1-10, preferably 1-8, and more preferably 1-3.

A library of KPI-bipodal peptide binder of the present invention having the above-described construct may be obtained by various methods known in the art. In this library, target binding region II of the KPI-bipodal peptide binder has a random sequence, which means that no sequence preference or no determined (or fixed) amino acid residue is placed at any position of target binding region II.

For example, the library of the KPI-bipodal peptide binder may be constructed by a split-synthesis method (Lam et al. (1991) Nature 354:82; and WO 92/00091), which is carried out on a solid supporter (e.g., polystyrene or polyacrylamide resin).

According to a preferable embodiment of the present invention, the library of the KPI-bipodal peptide binder is constructed by a cell surface display method (e.g., phage display, bacteria display, or yeast display). Preferably, the library of the KPI-bipodal peptide binder may be constructed by a display method on the basis of plasmids, bacteriophages, phagemids, yeasts, bacteria, mRNAs, or ribosomes.

Phage display is a technique that displays various polypeptides in a form of a protein fused with the coat protein on the phage surface (Scott, J. K. and Smith, G. P. (1990) Science 249: 386; Sambrook, J. et al., Molecular Cloning. A Laboratory Manual, 3rd ed. Cold Spring Harbor Press (2001); and Clackson and Lowman, Phage Display, Oxford University Press (2004)). A gene to be expressed is fused with gene III or gene VIII of filamentous phage (e.g., M13), thereby displaying random peptides.

A phagemid may be used in the phage display. The phagemid is a plasmid vector which has a replication origin of bacteria (e.g., ColE1) and one copy of intergenic region of bacteriophage. DNA fragments cloned into the phagemid are multiplied like a plasmid.

In cases where the library of the KIP-bipodal peptide binder is constructed by a phage display method, a preferable embodiment of the present invention includes the following steps of: (i) constructing an expression vector library including a fusion gene in which a gene coding a phage coat protein (e.g., gene III or gene VIII coat protein of the filamentous phage, such as M13) is fused with a gene coding a KPI-bipodal peptide binder, and a transcriptional regulatory sequence (e.g., lac promoter) operatively linked to the fusion gene; (ii) introducing the expression vector library into suitable host cells; (iii) culturing the host cells to form recombinant phage or phagemid virus particles to form recombinant phage or phagemid virus particles, thereby displaying a fusion protein on the phage surface; (iv) contacting the virus particles with a biological target molecule to bind the particles to the target molecule; and (v) separating the particles unbound to the target molecule.

The methods of constructing peptide libraries using phage display and screening the libraries are disclosed in U.S. Pat. Nos. 5,723,286, 5,432,018, 5,580,717, 5,427,908, 5,498,530, 5,770,434, 5,734,018, 5,698,426, 5,763,192, and 5,723,323.

The method of constructing an expression vector including a KPI-bipodal peptide binder gene may be carried out by the methods known in the art. For example, the expression vector may be constructed by inserting the KPI-bipodal peptide binder gene into a known phagemid or phage vector (e.g., pIGT2, fUSE5, fAFF1, fd-CAT1, m663, fdtetDOG, pHEN1, pComb3, pComb8, pCANTAB 5E (Pharmacia), LamdaSurfZap, pIF4, PM48, PM52, PM54, fdH, and p8V5).

Most phage display methods are carried out using filamentous phages, but lambda phage display (WO 95/34683; and U.S. Pat. No. 5,627,024), T4 phage display (Ren et al. (1998) Gene 215:439; and Zhu (1997) CAN 33:534) and T7 phage display (U.S. Pat. No. 5,766,905) may be used to construct a library of bipodal-peptide binder.

The method of introducing the vector library into suitable host cells may be carried out by various transformation methods, and may be carried out by, most preferably, electroporation (see, U.S. Pat. Nos. 5,186,800, 5,422,272, and 5,750,373). The host cells suitable in the present invention include cells of gram-negative bacteria, such as E. coli, and examples of suitable E. coli host include JM101, E. coli K12 strain 294, E. coli strain W3110, and E. coli XL-1Blue (Stratagene), but are not limited thereto. Preferably, the host cells are prepared using competent cells before transformation (Sambrook, J. et al., Molecular Cloning. A Laboratory Manual, 3rd ed. Cold Spring Harbor Press (2001)). The selection of transformed cells is carried out by culturing cells in media containing antibiotics (e.g., tetracycline and ampicillin). The selected transformed cells are further cultured in the presence of a helper phage to produce recombinant phages or phagemid virus particles. Suitable examples of the helper phage include Ex helper phage, M13-K07, M13-VCS, and R408, but are not limited thereto.

In the method of the present invention, step (c) may be carried out through various methods known in the art. First, clones binding to a target are selected from a library of KPI-bipodal peptide binder, and here, the selection may be carried out through a biopanning process (Sambrook, J. et al., Molecular Cloning. A Laboratory Manual, 3rd ed. Cold Spring Harbor Press (2001); and Clackson and Lowman, Phage Display, Oxford University Press (2004)). Then, the target affinities of the peptides produced from the colons selected through the biopanning process are analyzed. The target affinities may be analyzed by various methods known in the art, for example, bimolecular interaction analysis (BIA) (e.g., BIAcore™) (Sjolander & Urbaniczky, Anal. Chem. 63:23382345 (1991), Szabo et al., Curr. Opin. Struct. Biol. 5:699705 (1995); Smith E A, et al., Appl. Spectroscopy, 57:320A-332A (2003)), and the change in surface plasmon reasonance (SPR) may be used as an indicator for a real-time reaction between molecules.

According to a preferable embodiment of the present invention, the KPI-bipodal peptide binder selected in step (c) has a target affinity, increasing 2- to 10000-fold, more preferably, 2- to 5000-fold, 2- to 4000-fold, 2- to 3000-fold, 2- to 2000-fold, 50- to 10000-fold, 50- to 5000-fold, 50- to 4000-fold, 50- to 3000-fold, or 50- to 2000-fold, and still more preferably, 100- to 2000-fold, 200- to 2000-fold, 500- to 2000-fold, or 700- to 1500-fold. The increase in the target affinity in the present invention is very remarkable and unpredictable.

A preferable embodiment of the present invention, the KPI-bipodal peptide binder selected from step (c) has a dissociation constant (K_{D}) of 0.1-10000 nM with respect to the protein target.

According to a preferable embodiment of the present invention, the KPI-bipodal peptide binder selected from step (c) is an original bipodal peptide binder (BPB). The method further includes the following steps after step (c), and the target affinity of the initial BPB is increased by the following steps: (a′) constructing a first affinity BPB library by randomizing a sequence of any one of target binding region I and target binding region II of the initial BPB; (b′) selecting a first affinity optimization BPB molecule, which has an increased target affinity compared with the initial BPB, from the first affinity BPB library; (c′) constructing a second affinity BPB library by adding 1-10 random amino acids to a terminus of target binding region I, target binding region II, or target binding region I and target binding region II, of the first affinity optimization PBP molecule; and (d′) selecting a second affinity optimization BPB molecule, which has an increased target affinity as compared with the first affinity optimization BPB molecule, from the second affinity BPB library.

The method of increasing the target affinity of the BPB is described in detail below.

The KPI-bipodal peptide binder of the present invention not only has a use as a medicine but also may be used for detecting an in vivo material, in vivo molecule imaging, in vitro cell imaging, and targeting for drug delivery, and may be used as an escort molecule.

In accordance with still another aspect of the present invention, there is provided a nucleic acid molecule coding the KIP-bipodal peptide binder.

In accordance with another aspect of the present invention, there is provided a vector for expressing a bipodal peptide binder, the vector including a nucleic acid molecule coding a KIP-bipodal peptide binder.

In accordance with another aspect of the present invention, there is provided a transformant including a vector for expressing a KPI-bipodal peptide binder.

As used herein, the term “nucleic acid molecule” refers to comprehensively including DNA (gDNA and cDNA) and RNA molecules, and the nucleotide as a basic constituent unit in the nucleic acid molecule includes natural occurring nucleotides, and analogues with modified sugars or bases (Scheit, Nucleotide Analogs, John Wiley, New York (1980); Uhlman, and Peyman, Chemical Reviews, 90:543-584 (1990)).

According to a preferable embodiment of the present invention, the vector of the present invention includes not only the nucleic acid molecule coding a KPI-bipodal peptide binder but also a strong promoter (e.g., tac promoter, lac promoter, lacUV5 promoter, lpp promoter, p_{L}^{λ} promoter, p_{R}^{λ} promoter, rac5 promoter, amp promoter, recA promoter, SP6 promoter, trp promoter, T7 promoter, etc.) capable of performing transcription in the nucleic acid molecule, a ribosome-binding site for initiation of translation, and transcription/translation termination sequences.

According to a preferable embodiment of the present invention, the vector of the present invention may further include a signal sequence (e.g., pelB) at the 5-direction of the nucleic acid molecule coding a KPI-bipodal peptide binder. In addition, according to a preferable embodiment of the present invention, the vector of the present invention further includes a tagging sequence (e.g., myc tag) for confirming whether the KPI-bipodal peptide binder is favorably expressed on the phage surface.

According to a preferable embodiment of the present invention, the vector of the present invention includes a phage coat protein, preferably a gene coding a gene III or gene VIII coat protein of filamentous phage, such as M13. According to a preferable embodiment of the present invention, the vector of the present invention includes a replication origin (e.g., ColE1) of bacteria and/or a replication origin of bacteriophage. Meanwhile, the vector of the present invention may include an antibiotic-resistant gene that is normally used in the art, as a selective label, and may include resistant genes against ampicillin, gentamycin, carbenicillin, chloramphenicol, streptomycin, kanamycin, geneticin, neomycin, and tetracycline.

The transformant of the present invention preferably includes gram-negative bacteria cells such as E. coli, and examples of the suitable E. coli host may include JM101, E. coli K12 strain 294, E. coli strain W3110, and E. coli XL-1Blue (Stratagene), but are not limited thereto. The method of delivering the vector of the present invention into a host cell may be carried out by a CaCl₂ method (Cohen, S. N. et al., Proc. Natl. Acac. Sci. USA, 9:2110-2114(1973)), other methods (Cohen, S. N. et al., Proc. Natl. Acac. Sci. USA, 9:2110-2114(1973); and Hanahan, D., J. Mol. Biol., 166:557-580(1983)), and electroporation methods (U.S. Pat. Nos. 5,186,800, 5,422,272, and 5,750,373).

The KPI-bipodal peptide binder of the present invention improves or modifies the known peptide in view of the target affinity, to exhibit a K_{D} value (dissociation constant) of a very low level (e.g., nM level), and thus provides a peptide exhibiting a very high affinity to a biological target molecule.

Therefore, the KPI-bipodal peptide binder technique of the present invention can significantly improve applicability of the known peptide.

In accordance with an aspect of the present invention, there is provided a method for increasing the target affinity of a bipodal peptide binder (BPB), the method including:

(a) constructing a first affinity BPB library by randomizing a sequence of any one of target binding region I and target binding region II of the initial BPB, wherein the initial BPB includes: (i) a structure stabilizing region including parallel, antiparallel, or parallel and antiparallel amino acid strands with interstrand non-covalent bonds; and (ii) target binding region I and target binding region II bound to both termini of the structure stabilizing region, respectively, and including m and n amino acids, respectively, which are randomly selected;

(b) selecting a first affinity optimization BPB molecule, which has an increased target affinity compared with the initial BPB, from the first affinity BPB library;

(c) constructing a second affinity BPB library by adding 1-10 random amino acids to a terminus of target binding region I, target binding region II, or target binding region I and target binding region II, of the first affinity optimization PBP molecule; and

(d) selecting a second affinity optimization BPB molecule, which has an increased target affinity as compared with the first affinity optimization BPB molecule, from the second affinity BPB library.

In accordance with an aspect of the present invention, there is provided a bipodal peptide binder (BPB) having a target affinity which is increased by the method.

The present inventors have endeavored to develop a technique capable of optimizing the target affinity of the bipodal peptide binder conventionally developed by themselves. As a result, the present inventors have developed a method capable of very effectively optimizing or maximizing the target affinity of BPB while maintaining the whole structure of BPB.

As used herein to cite the target affinity, the term “optimization” is used in the same meaning of maximization, unless otherwise described.

The present invention basically adopts a structure of BPB. BPB is an affinity peptide that was first proposed by the present inventors, and see WO 2010/047515 for the detailed description of the peptide.

The method for optimizing the target affinity of BPB of the present invention has overlapping contents with the method for improving the target affinity of a peptide as described above, and descriptions of the overlapping contents therebetween are omitted to avoid excessive complications of the specification.

Specific examples of the original bipodal peptide binder (BPB) used in the present invention are described in SEQ ID NOs: 20 to 38 and 40 to 41.

According to the present invention, in the foregoing original bipodal peptide binder (BPB) structure, the sequence of any one of target binding region I and target binding region II is randomized to construct a first affinity BPB library.

The first affinity BPB library may be constructed by various methods known in the art.

In this library, the first affinity BPB library has random sequences, and this means that no sequence preference or no determined (or fixed) amino acid residue is placed at any position of target binding region I and/or target binding region II.

For example, the first affinity BPB library may be constructed by a split-synthesis method (Lam et al. (1991) Nature 354:82; WO 92/00091), which is carried out on a solid supporter (e.g., polystyrene or polyacrylamide resin).

According to a preferable embodiment of the present invention, the library of the bipodal peptide binder is constructed by a cell surface display method (e.g., phage display, bacteria display, or yeast display). Preferably, the library of the bipodal peptide binder may be constructed by a display method on the basis of plasmids, bacteriophages, phagemids, yeasts, bacteria, mRNAs, or ribosomes.

In cases where the first BPB library is constructed by a phage display method, a preferable embodiment of the present invention includes the following steps of: (i) constructing an expression vector library including a fusion gene in which a gene coding a phage coat protein (e.g., gene III or gene VIII coat protein of the filamentous phage, such as M13) is fused with a gene coding a KPI-bipodal peptide binder, and a transcriptional regulatory sequence (e.g., lac promoter) operatively linked to the fusion gene; (ii) introducing the expression vector library into suitable host cells; (iii) culturing the host cells to form recombinant phage or phagemid virus particles to form recombinant phage or phagemid virus particles, thereby displaying a fusion protein on the phage surface; (iv) contacting the virus particles with a biological target molecule to bind the particles to the target molecule; and (v) separating the particles unbound to the target molecule.

The methods of constructing the first affinity BPB libraries using phage display and screening the libraries are disclosed in U.S. Pat. Nos. 5,723,286, 5,432,018, 5,580,717, 5,427,908, 5,498,530, 5,770,434, 5,734,018, 5,698,426, 5,763,192, and 5,723,323.

An expression vector including the first affinity BPB library may be constructed by a method known in the art. For example, an expression vector may be constructed by inserting a bipodal peptide binder into a known phagemid or phage vector (e.g., pIGT2, fUSE5, fAFF1, fd-CAT1, m663, fdtetDOG, pHEN1, pComb3, pComb8, pCANTAB 5E (Pharmacia), LamdaSurfZap, pIF4, PM48, PM52, PM54, fdH, and p8V5).

After the first affinity BPB library is constructed, a first affinity optimization BPB molecule, of which the target affinity is increased as compared with the original BPB, is selected from the first affinity BPB library. First, clones having increased target affinities as compared with the original BPB are selected from the first affinity BPB library, and here, the selection may be carried out through a biopanning process (Sambrook, J. et al., Molecular Cloning. A Laboratory Manual, 3rd ed. Cold Spring Harbor Press (2001); Clackson and Lowman, Phage Display, Oxford University Press (2004)). Then, target affinities of the peptides produced from the clones selected through the biopanning process are analyzed. The target affinity may be analyzed by various methods known in the art, for example, bimolecular interaction analysis (BIA) (e.g., BIAcore™) (Sjolander & Urbaniczky, Anal. Chem. 63:23382345(1991), Szabo et al., Curr. Opin. Struct. Biol. 5:699705(1995); Smith E A, et al., Appl. Spectroscopy, 57:320A-332A(2003)), and the change in surface plasmon reasonance (SPR) may be used as an indicator for a real-time reaction between molecules.

After the first affinity optimization BPB molecule is selected, a second affinity BPB library is constructed by adding 1-10 random amino acids to the terminus of target binding region I, target binding region II, or target binding region I and target binding region II of the first affinity optimization BPB molecule.

The second affinity BPB library may be constructed by the foregoing method of constructing the first affinity BPB library.

In the construction of the second affinity BPB library, the number of random amino acids added is 1-10, preferably 1-5, more preferably 1-3, and still more preferably 1-2.

After the second affinity BPB library is constructed, a second affinity optimization BPB molecule, of which the target affinity is increased as compared with the first affinity optimization BPB molecule, is selected from the second affinity BPB library. The second affinity optimization BPB molecule may be selected by the same method as in the selection of the first affinity optimization BPB molecule.

According to a preferable embodiment of the present invention, the second affinity optimization BPB molecule exhibits a target affinity, which increases 2- to 40-fold compared with the original BPB. The second affinity optimization BPB molecule exhibits a target affinity, increasing more preferably 5- to 40-fold, still more preferably 10- to 35-fold, and still more preferably 20- to 25-fold, compared with the original BPB.

According to a preferable embodiment of the present invention, the second affinity optimization BPB molecule has a dissociation constant (K_{D}) of 0.1-20 nM with respect to a target. The second affinity optimization BPB molecule has a dissociation constant (K_{D}) of more preferably 0.1-10 nM, and still more preferably 1-8 nM, with respect to a target.

That is, the BPB, of which the target affinity is optimized by the method of the present invention, can have a very high affinity to the target.

In accordance with another aspect of the present invention, there is provided a bipodal peptide binder (BPB) having a target affinity which is increased by the method of the present invention.

In accordance with still another aspect of the present invention, there is provided a nucleic acid molecule coding the bipodal peptide binder (BPB) having an increased target affinity.

### Advantageous Effects

Features and advantages of the present invention are summarized as follows.

a) The present invention provides a method capable of remarkably improving the target affinity of a known peptide, and the method is very efficient and simple.

(b) The present invention can allow the target affinity of a known peptide to increase 1000-fold.

(c) The KPI-bipodal peptide binder technique of the present invention can significantly improve applicability of the known peptide.

(d) The present invention provides a method for increasing the target affinity of a bipodal peptide binder (BPB), and the method is very efficient and simple.

(e) The present invention can allow the target affinity of BPB to increase 20- to 25-fold.

(f) The BPB, of which the target affinity is increased by the method of the present invention, exhibits a very low level (e.g., nM level) of K_{D} value (dissociation constant), and thus exhibits a very high affinity to a target.

### BRIEF DESCRIPTION OF THE DRAWINGS

FIG. 1 is a schematic view of a KPI-bipodal peptide binder technique of the present invention, which increases the target affinity of the conventional known peptide.

FIG. 2 shows screening results for improving the affinity of a known peptide binding to streptavidin.

FIGS. 3a and 3b show colony ELISA results after the affinity to target streptavidin is improved by the KIP-BPB technique of the present invention.

FIGS. 4a and 4b show analysis results of target specificities of Strep_opti1 and Strep_opti2, as KPI-BPBs, with respect to streptavidin, which are selected by the KPI-BPB technique of the present invention.

FIG. 5 shows analysis results of a target affinity of Strep_opti1, as a KPI-BPB, with respect to streptavidin, which is selected by the KPI-BPB technique of the present invention.

FIG. 6 shows screening results for improving the affinity of a known peptide binding to VEGFR1.

FIG. 7 shows colony ELISA results after the affinity to target VEGFR1 is improved by the KIP-BPB technique.

FIG. 8 shows analysis results of target specificities of VEGFR1_opti1, VEGFR1_opti2, and VEGFR1_opti3, as KPI-BPBs, with respect to VEGFR1, which are selected by the KPI-BPB technique.

FIG. 9 shows analysis results of a target affinity of VEGFR1_opti1, as a KPI-BPB, with respect to VEGFR1, which is selected by the KPI-BPB technique.

FIG. 10 is a schematic view of the original bipodal peptide binder (BPB).

FIG. 11 is a schematic view showing an optimizing procedure of a BPB according to the present invention.

FIG. 12 shows colony ELISA results after the optimization of BPB1_{EDB}.

FIG. 13 shows measurement results of target affinities of three first affinity optimization BPB molecules selected from a first affinity BPB library constructed after the first optimization.

FIG. 14 is a graph showing an output/input phage ratio of biopanning using a BPB3 optimization library, which is a second affinity BPB library constructed after the second optimization.

FIG. 15 is a graph showing an output/input phage ratio of biopanning using a BPB4 optimization library, which a second affinity BPB library constructed after the second optimization.

FIG. 16 shows multiple alignments of amino acid sequences obtained through target affinity screening on peptides in the BPB3 optimization library, which is a second affinity BPB library constructed after the second optimization.

FIG. 17 shows multiple alignments of amino acid sequences obtained through target affinity screening on peptides in the BPB4 optimization library, which a second affinity BPB library constructed after the second optimization.

FIG. 18 shows affinity measurement results of the second affinity optimization BPB molecule, BPB3.

FIG. 19 shows affinity measurement results of the second affinity optimization BPB molecule, BPB4.

FIG. 20 shows target binding kinetic data of BPB1, BPB2, BPB3, and BPB4 at 250 nM.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### Example I

### Preparation of Affinity Optimization Peptide

### Method

### Construction of BPB2_{EDB} Optimization Library

In order to construct a BPB2_{EDB} optimization library, two oligonucletides (5′-TTC TAT GCG GCC CAG CTG GCC (NNK)₆ GGA TCT TGG ACA TGG GAA AAC GGA AAA-3′; and 5′-AAC AGT TTC TGC GGC CGC TCC TCC TCC TTG CTC CAA CCT AAT AAT TCC CTT CCA TGT CCA TTT TCC GTT-3′) (BPB2_{EDB}_{_{—}}F1) and (5′-AAC AGT TTC TGC GGC CGC TCC TCC TCC TTG CTC CAA CCT AAT AAT TCC CTT CCA TGT CCA TTT TCC GTT-3′) (BPB2_{EDB}_{_{—}}B1), (N=A, T, G, or C); (K=G or T); (M=C or A) were synthesized. In order to prepare a double strand, BPB2_{EDB}_{_{—}}F1 4 μM (final concentration), BPB2_{EDB}_{_{—}}B1 4 μM (final concentration), 2.5 mM dNTP mix 4 μl, Ex TaqDNA polymerase 1 μl (10 U) (Takara, Seoul, Korea), and 10×PCR buffer 5 μl were mixed to prepare 25 mixture solutions having a total of 50 μl through addition of distilled water. These mixture solutions were subjected to PCR (94° C. 5 min, 60 cycles: 30° C. 30 seconds, and 72° C. 30 seconds, and 72° C. 7 min) to prepare double strands, which were then purified using a PCR purification kit (GeneAll, Seoul, Korea). In order to ligate the BPB2_{EDB} insert gene to the pIGT2 phagemid vector (Ig therapy, Chuncheon, Korea), the insert gene and the pIGT2 phagemid vector were treated with restriction enzymes. About 11 μg of insert DNA was allowed to react with SfiI (NEB) and NofI (NEB) for 4 hrs, respectively, followed by purification using a PCR purification kit. Thereafter, about 40 μg of the pIGT2 phagemid vector was allowed to react with SfiI (NEB) and NofI (NEB) for 4 hrs, respectively, and then reacted with calf intestinal alkaline phosphatase (CIP; NEB, Ipswich) for 1 hr, followed by purification using a PCR purification kit. Then, 2.9 μg of the insert gene, which was obtained by quantifying the reaction products using a UV-visible light spectrophotometer (Ultrospec 2100 pro, Amersham Bioscience), was ligated to 12 μg of the pIGT2 phagemid vector at 18° C. for 15 hrs using T4 DNA ligase (Bioneer, Daejeon, Korea), followed by precipitation using ethanol, and then DNA was lysed with 100 μl of TE buffer. E. coli XL-1 competent cells were transformed with the resultant material by electroporation, finally constructing an 8×10⁷ library.

Construction of Affinity Improvement Libraries of Streptavidin-Binding Peptide WSHPQFEK and VEGFR1 (GNQWFI)

It was known in 1996 that streptavidin-binding peptide WSHPQFEK has an affinity of 72 μM and specifically binds to a streptavidin protein. It was reported in 2005 that vascular endothelial growth factor receptor 1 (VEGFR1)-binding peptide GNQWFI specifically binds to a VEGFR1 protein. Here, in order to improve affinities of these peptides, affinity improvement libraries were designed and constructed. In order to construct a streptavidin maturation library, two oligonucleotides (5′-TTC TAT GCG GCC CAG CTG GCC TGG AGC CAT CCG CAG TTT GAA AAA GGC GGA GGA TCT TGG ACA TGG GAA AAC GGA AAA-3′) (F1) and (5′-AAC AGT TTC TGC GGC CGC TCC TCC TCC MNN MNN MNN MNN MNN MNN TCC CTT CCA TGT CCA TTT TCC GTT-3′) (B1), (N=A, T, G, or C); (K=G or T); (M=C or A) were synthesized. In order to construct a VEGFR1 maturation library, two oligonucleotides (5′-TTC TAT GCG GCC CAG CTG GCC GGC AAC CAG TGG TTT ATT GGA GGA GGA TCT TGG ACA TGG GAA AAC GGA AAA-3′) (F1) and (5′-AAC AGT TTC TGC GGC CGC TCC TCC TCC MNN MNN MNN MNN MNN MNN TCC CTT CCA TGT CCA TTT TCC GTT-3′) (B1), (N=A, T, G, or C); (K=G or T); (M=C or A) were synthesized. In order to prepare a double strand of each peptide, F1 4 μM (final concentration), B1 4 μM (final concentration), 2.5 mM dNTP mix 4 μl, Ex TaqDNA polymerase 1 μl (10 U) (Takara, Seoul, Korea), and 10×PCR buffer 5 μl were mixed to prepare 25 mixture solutions having a total of 50 μl through addition of distilled water. These mixture solutions were subjected to PCR (94° C. 5 min, 60 cycles: 30° C. 30 seconds, and 72° C. 30 seconds, and 72° C. 7 min), thereby preparing double strands, which were then purified using a PCR purification kit (GeneAll, Seoul, Korea). In order to ligate each of two different insert genes for the maturation libraries to the pIGT2 phagemid vector (Ig therapy, Chuncheon, Korea), each of the insert gene and the pIGT2 phagemid vector were treated with restriction enzymes. About 10 μg of insert DNA was allowed to react with SfiI (NEB) and NofI (NEB) for 4 hrs, respectively, followed by purification using a PCR purification kit. Thereafter, about 40 μg of the pIGT2 phagemid vector was allowed to react with SfiI (NEB) and NofI (NEB) for 4 hrs, respectively, and then reacted with calf intestinal alkaline phosphatase (CIP; NEB, Ipswich) for 1 hr, followed by purification using a PCR purification kit. Then, 3 μg of the insert gene, which was obtained by quantifying the reaction products using a UV-visible light spectrophotometer (Ultrospec 2100 pro, Amersham Bioscience), was ligated to 12 μg of the pIGT2 phagemid vector at 18° C. for 15 hrs using T4 DNA ligase (Bioneer, Daejeon, Korea), followed by precipitation using ethanol, and then DNA was lysed with 100 μl of TE buffer. E. coli XL-1 competent cells were transformed with the resultant material by electroporation, finally constructing 6×10⁷ and 5×10⁷ libraries.

### Biopanning for Finding Streptavidin-Binding Peptide and VEGFR1-Binding Peptide

Streptavidin (10 μg/ml) and BSA (10 μg/ml) were added to wells of a 96-well ELISA plate (50 μl per well for each), and then allowed to stand at 4° C. overnight. Then, the wells were washed three times with 0.1% PBST, and blocked with 2% BSA diluted with PBS at room temperature for 2 hrs. Then, the whole solution was discarded, and then the plate was washed three times with 0.1% PBST.

800 μl of a solution containing streptavidin maturation recombinant phages and 200 μl of 10% BSA were added thereto, and in order to remove phages binding to BSA, the mixture was put in BSA-coated wells and allowed to stand at 27° C. for 1 hr. The supernatant was collected, and then allowed to react with the streptavidin protein at 27° C. for 1 hr. Then, the wells were washed ten times with 0.05% PBST, and then the phages were subsequently eluted for 20 min by adding 1 ml of 0.2 M glycine/HCl (pH 2.2) to each well (50 μl per well). 1 ml of phages were collected in a tube, and neutralized by adding 150 μl of 2 M Tris-base (pH 9.0).

In order to count input phages and elute phages for each biopanning step, the phages were mixed with XL-1 BLUE cells (OD=0.7), and the mixture was plated on agar media containing ampicillin. In order to repeat panning, the phages were mixed with 10 ml of E. coli ER cells, and stirred and cultured at 37° C. for 1 hr at a rate of 200 rpm. After mixing with ampicillin (50 μg/ml) and 20 mM glucose, 2×10¹⁰ pfu of Ex helper phages were added, followed by stirring and culturing at 37° C. for 1 hr at a rate of 200 rpm. After the culture liquid was centrifuged at 1,000×g for 10 min to remove the supernatant, the precipitated cells were resuspended in 40 ml LB liquid media containing ampicillin (50 μg/ml) and kanamycin (25 μg/ml), and then the solution was stirred and cultured at a rate of 200 rpm at 30° C. overnight. The culture liquid was centrifuged at 4,000×g for 10 min at 4° C. 8 ml of 5×PEG/NaCl [20% PEG(w/v) and 15% NaCl(w/v)] was added to the supernatant, and then the mixture was allowed to stand at 4° C. for 1 hr. After centrifugation, the PEG solution was completely removed, and the phage peptide pellets were lysed with 1 ml PBS solution, which was then used in the second biopanning. Each panning step was carried out by the same method as described above except that the number of times of washing with 0.1% PBST increased 10 times, 20 times, and 30 times by steps. Biopanning of VEGFR1 was also performed as described above. Each panning step was carried out by the same method as described above except that the number of washing with 0.1% PBST increased 10 times, 20 times, and 30 times by steps.

### Search of Phage Peptides Specific to Streptavidin and VEGFR1 Proteins (Phage ELISA)

E. coli ER cells were infected with the phages collected in the fourth biopanning step from the streptavidin maturation library, and then the cells were plated such that the number of plaques per plate is 100-200. Thereafter, 60 plaques were inoculated in 2 ml of LB-ampicillin (50 μg/ml) media using a sterile tip, and subjected to shaking culture at 37° C. for 5 hrs. 5×10⁹ pfu (OD=0.8-1) of Ex helper phages were added, followed by stirring and culturing at a rate of 200 rpm for 1 hr at 37° C. After the culture liquid was centrifuged at 1,000×g for 10 min to remove the supernatant, the precipitated cells were resuspended in 1 ml LB liquid media containing ampicillin (50 μg/ml) and kanamycin (25 μg/ml), and then the solution was stirred and cultured at a rate of 200 rpm at 30° C. overnight. The culture liquid was centrifuged at 10,000×g for 20 min at 4° C. to collect the supernatant, and then 2% skim milk was added, which was then used in the search of phage peptides.

10 μg/ml streptavidin was put in 30 wells (50 μl per well) of a 96-well ELISA plate, and 10 μg/ml BSA was put in 30 wells (50 μl per well), followed by standing at 4° C. overnight. Thereafter, all the wells were washed three times with 0.1% PBST, and blocked at room temperature for 2 hrs using 2% skim milk diluted with PBS. Then, the solution was discarded, followed by washing with 0.1% PBST three times. 100 μl of the phage peptide solution amplified for each clone was dispensed into the wells to which the streptavidin protein adheres and the wells to which the BSA protein adheres, and allowed to stand at 27° C. for 1.5 hrs. After washing ten times with 0.1% PBST solution, the HRP-conjugated anti-M13 antibody (GE Healthcare) was diluted to 1:1,000, and then the reaction was performed at 27° C. for 1 hr. After washing five times with 0.1% PBST, 100 μl of the TMB solution was dispensed into each well to induce a colorimetric reaction, and then the reaction was stopped by adding 100 μl of 1 M HCl. The absorbance was measured at 450 nm to select clones in which the absorbance of streptavidin was 20-fold higher than that of BSA. XL1 cells were infected with these phages, and the cells were plated such that the number of plaques per plate is 100-200. Thereafter, the plaques were inoculated in 4 ml of LB-ampicillin (50 μg/ml) culture liquid using a sterile tip, and subjected to shaking culture at 37° C. for one day. The plasmids were purified using a plasmid preparation kit, and then sequencing was commissioned (Genotech, Daejeon, Korea). As a sequencing primer, 5′-GATTACGCCAAGCTTTGGAGC-3′, which is a vector sequence, was used. The same method as described above was also performed for VEGFR1.

### Experiment on Specificity of Phages Binding to Streptavidin Protein

In order to investigate specificities to streptavidin, of peptides (Strep_opti1 and Strep_opti2) obtained through sequencing, recombinant phages expressing these peptides were used to investigate specificities to streptavidin, BSA, ovalbumin, and VEGF. First, 5 μg/ml streptavidin, BSA, ovalbumin, and VEGF were put in a 96-well ELISA plate (50 μl per well), and then allowed to stand at 4° C. overnight. Next day, all the wells were washed three times with 0.1% PBST, and blocked at room temperature for 2 hrs using 2% BSA diluted with PBS. Then, the solution was discarded, followed by washing with 0.1% PBST three times. 800 μl of recombinant phages of each of Strep_opti1 and Strep_opti2 and 200 μl of 10% BSA were added thereto, and each 100 μl was dispensed into streptavidin, BSA, ovalbumin, and VEGF wells, followed by standing at 27° C. for 1 hr. After washing five times with 0.1% PBST solution, the HRP-conjugated anti-M13 antibody (GE Healthcare) was diluted to 1:1,000, and then the reaction was performed at 27° C. for 1 hr. After washing five times with 0.1% PBST, 100 μl of the TMB solution was dispensed into each well to induce a colorimetric reaction, and then the reaction was stopped by adding 100 μl of 1 M HCl. The absorbance was measured at 450 nm.

### Experiment on Specificity of Phages Binding to VEGFR1 Protein

In order to investigate specificities to streptavidin, of peptides (VEGFR1_opti1, VEGFR1_opti2, and VEGFR1_opti3) obtained through sequencing, recombinant phages expressing these peptides were used to investigate specificities to VEGFR1, VEGFR2, streptavidin, HSA, TNF, and VEGF. First, 5 μg/ml VEGFR1, VEGFR2, streptavidin, HSA, TNF, and VEGF were put in a 96-well ELISA plate (50 μl per well), and then allowed to stand at 4° C. overnight. The next day, all the wells were washed three times with 0.1% PBST, and blocked at room temperature for 2 hrs using 2% BSA diluted with PBS. Then, the whole solution was discarded, followed by washing three times with 0.1% PBST. 800 μl of recombinant phages of each of VEGFR1_opti1, VEGFR1_opti2, and VEGFR1_opti3 and 200 μl of 10% BSA were added thereto, and each 100 μl was dispensed into VEGFR1, VEGFR2, streptavidin, HSA, TNF, and VEGF wells, followed by standing at 27° C. for 1 hr. After washing five times with 0.1% PBST solution, the HRP-conjugated anti-M13 antibody (GE Healthcare) was diluted to 1:1,000, and then the reaction was performed at 27° C. for 1 hr. After washing with 0.1% PBST five times, 100 μl of the TMB solution was dispensed into each well to induce a colorimetric reaction, and then the reaction was stopped by adding 100 μl of 1 M HCl. The absorbance was measured at 450 nm.

### Binding Assay of Strep_Opti1 and Control Peptide (SPR)

A streptavidin-binding peptide optimized peptide and an existing peptide were synthesized (Anygen). The affinity measurement was conducted using BIAcore X (Biacore AB, Uppsala, Sweden). Here, 2000 RU of streptavidin was allowed to flow on the CM5 chip (Biacore) for immobilization. PBS (pH 7.4) was used as a running buffer, and the kinetics at different concentrations was measured under a flow rate of 30 μl per minute. The affinity was calculated using BlAevaluation software (Biacore AB, Uppsala, Sweden).

### Binding Assay of VEGFR1_Opti1 and Control Peptide (SPR)

A VEGFR1-binding peptide optimized peptide and an existing peptide were synthesized (Anygen). The affinity measurement was conducted using BIAcore X (Biacore AB, Uppsala, Sweden). Here, 7000 RU of VEGFR1 was allowed to flow on the CM5 chip (Biacore) for immobilization. PBS (pH 7.4) was used as a running buffer, and the kinetics at different concentrations was measured under a flow rate of 30 μl per minute. The affinity was calculated using BlAevaluation software (Biacore AB, Uppsala, Sweden).

### Results

### Design of Method for Improving Affinity of Existing Peptide

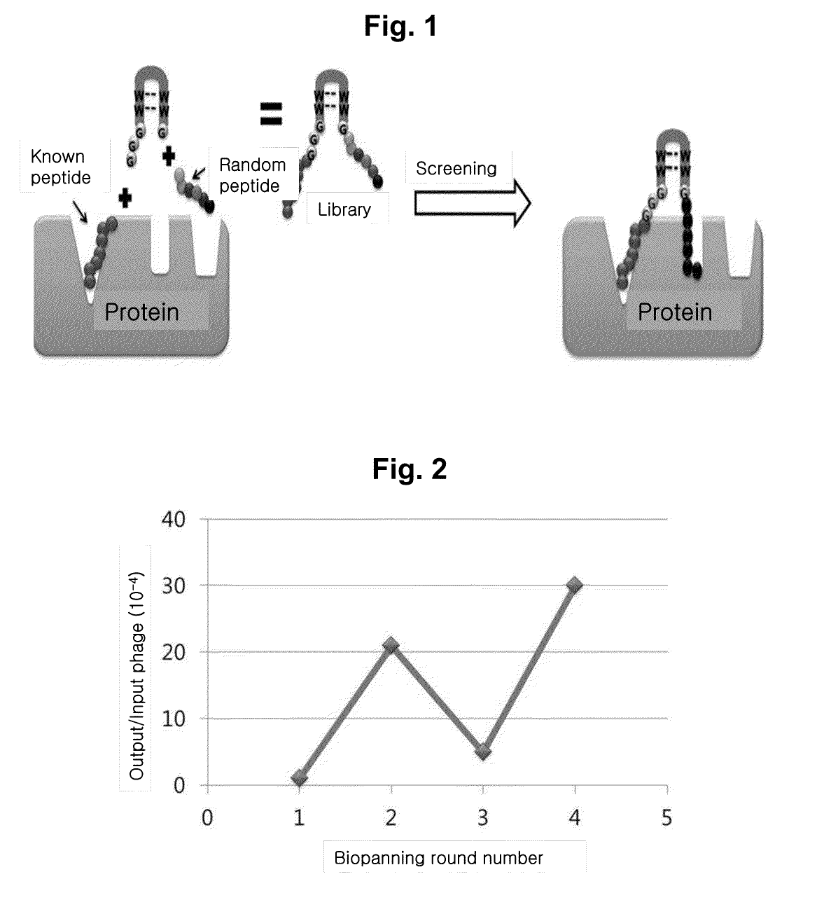

Most of the known peptides have restrictions in several applications due to low target affinities thereof. The present invention suggests a novel and innovative method for improving affinities of existing peptides. As shown in FIG. 1, a peptide, which binds to a new binding region adjacent to a region of a target, to which a known peptide (reported linear peptide) binds, was found and then the known peptide and the new binding region-binding peptide are linked to a structure stabilizing scaffold, thereby inducing 100- to 1000-fold improvement in affinity.

### Construction of Streptavidin Maturation and VEGFR1 Maturation Libraries

As an example, phage libraries were constructed using a streptavidin-binding peptide (WSHPQFEK) as a known peptide, and a VEGFR1-binding peptide (GNQWFI)

Optimization library of streptavidin-binding peptide: WSHPQFEKGGGSWTWENGKWTWKGXXXXXX (X=random amino acid); Optimization library of VEGFR1-binding peptide: GNQWFIGGGSWTWENGKWTWKGXXXXXX (X=random amino acid).

### Screening of Optimization of Streptavidin-Binding Peptide

In order to induce the improvement in affinity of the streptavidin-binding peptide (WSHPQFEK), the above phage library was constructed, and screening was carried out through the library. FIG. 2 compares output phage/input phage counts in the screening procedure for improving the affinity of the streptavidin-binding peptide. As a result of observation after the fourth biopanning, the output phage/input phage ratio was shown to increase 30-fold compared with the first biopanning.

Phage ELISA after Optimizing Affinity of Streptavidin-Binding Peptide

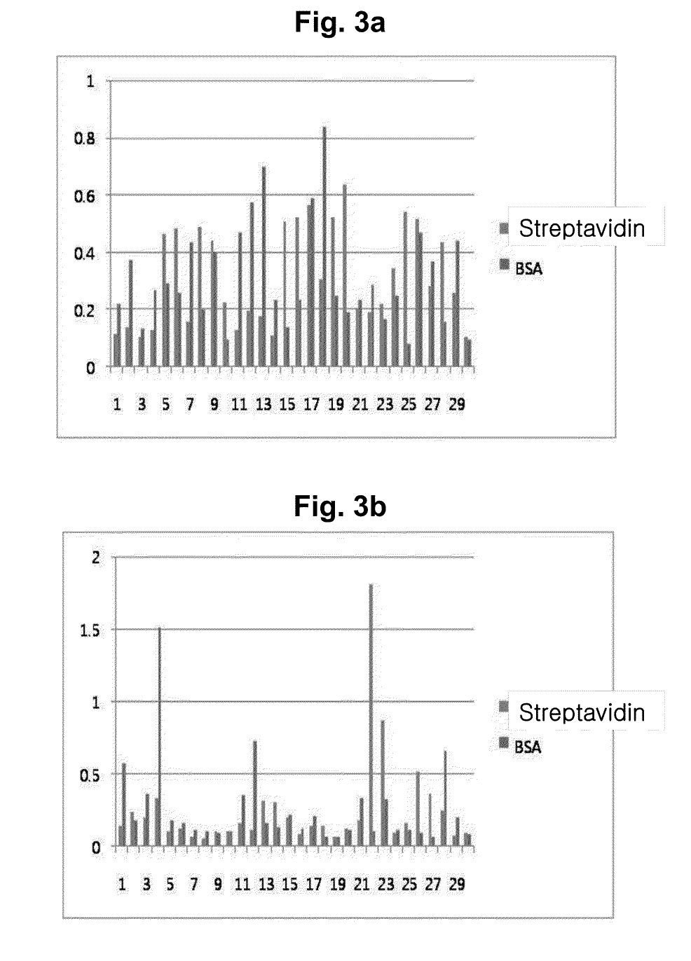

Phages, which were collected from the last panning step of the affinity improvement library of the streptavidin-binding peptide, were secured in a plaque form. After 60 phages were amplified from each plaque, ELISA was performed on streptavidin and BSA (FIGS. 3a and 3b). DNA sequencing was performed on 16 clones in which the aborbance of BSA was at least 3-fold higher than that of streptavidin. Two repeated peptide sequences were obtained: Strep_opti1: WSHPQFEKGGGSWTWENGKWTWKG AHPQVR (SEQ ID NO: 42); Strep_opti2: WSHPQFEKGGGSWTWENGKWTWKG TLIHPM (SEQ ID NO: 43)

### Investigation on Specificities of Strep_Opti1 and Strep_Opti2

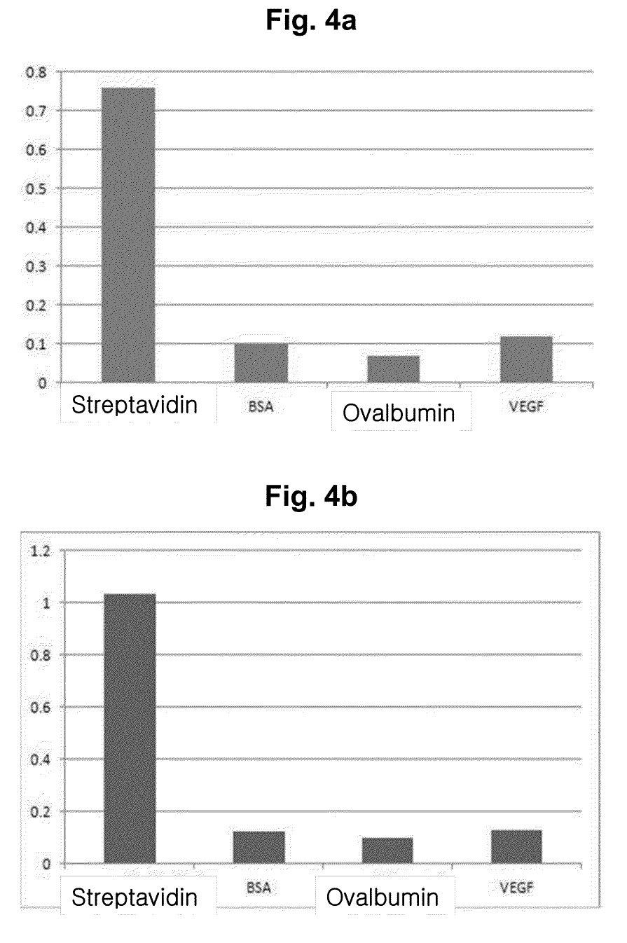

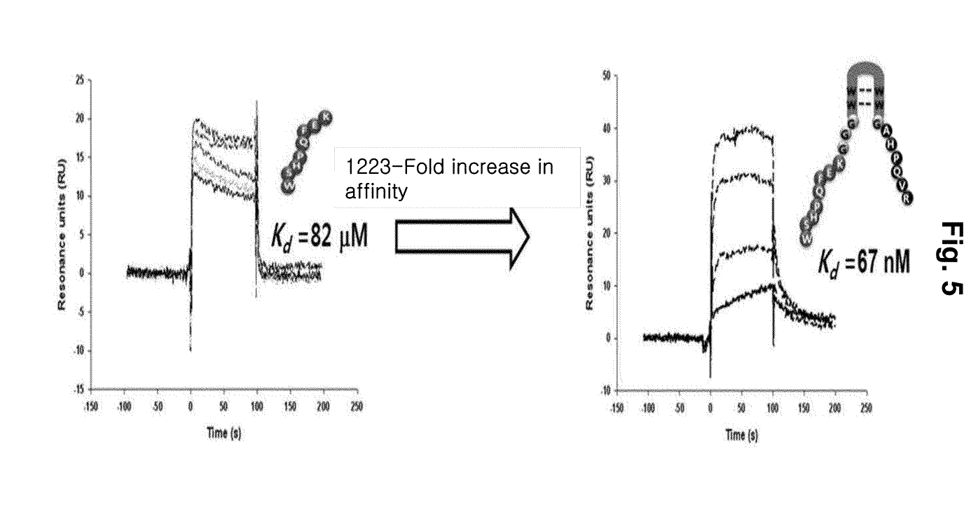

In order to investigate specificities of Strep_opti1 and Strep_opti2 as optimized peptides, ELISA was performed on streptavidin, BSA, ovalbumin, and VEGF. It can be seen from FIGS. 4a and 4b that the two peptides specifically bind to streptavidin.

### Affinity Measurement

Optimized peptide Strep_opti1 and control peptide WSHPQFEK were synthesized, and affinities thereof were measured using the SPR Biacore system. As a result of affinity measurement, Strep_opti1 with an optimized affinity had a K_{D} value of 67 nM, which showed a 1223-fold increase compared with control peptide WSHPQFEK having a K_{D} value of 82 μM (FIG. 5).

Therefore, it can be seen that the affinity can be easily optimized by at least 1000 times through the method suggested in the present invention.

### Screening of Optimization of VEGFR1-Binding Peptide

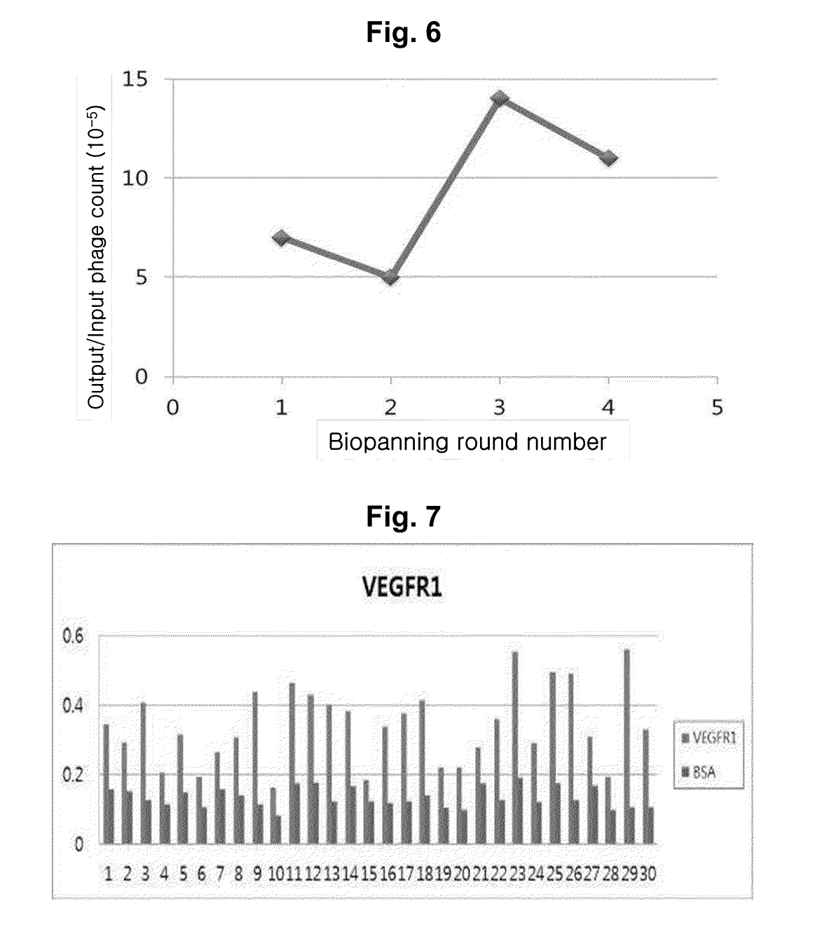

In order to induce the improvement in affinity of the VEGFR1-binding peptide (GNQWFI), the above phage library was constructed, and screening was carried out through the library. FIG. 6 compares output phage/input phage counts during the screening procedure for improving affinity of the VEGFR1-binding peptide. As a result of observation after the fourth biopanning, the output phage/input phage ratio was shown to increase by about 2 times compared with the first biopanning.

Phage ELISA after Optimizing Affinity of VEGFR1-Binding Peptide

Phages, which were collected from the last panning step of the affinity improvement library of the VEGFR1-binding peptide, were secured in a plaque form. After 30 phages were amplified from each plaque, ELISA was performed on VEGFR1 and BSA (FIG. 7). DNA sequencing was performed on 12 clones in which the absorbance of BSA was at least 3-fold higher than that of VEGFR1. Three repeated peptide sequences were obtained: VEGFR1_opti1: GNQWFIGGGSWTWENGKWTWKGRIPKNR (SEQ ID NO: 44); VEGFR1_opti2: GNQWFIGGGSWTWENGKWTWKGKLQNPM (SEQ ID NO: 45); VEGFR1_opti3: GNQWFIGGGSWTWENGKWTWKGQQAIQP (SEQ ID NO: 46).

### Investigation on Specificities of VEGFR1_Opti1, VEGFR1_Opti2, and VEGFR1_Opti3

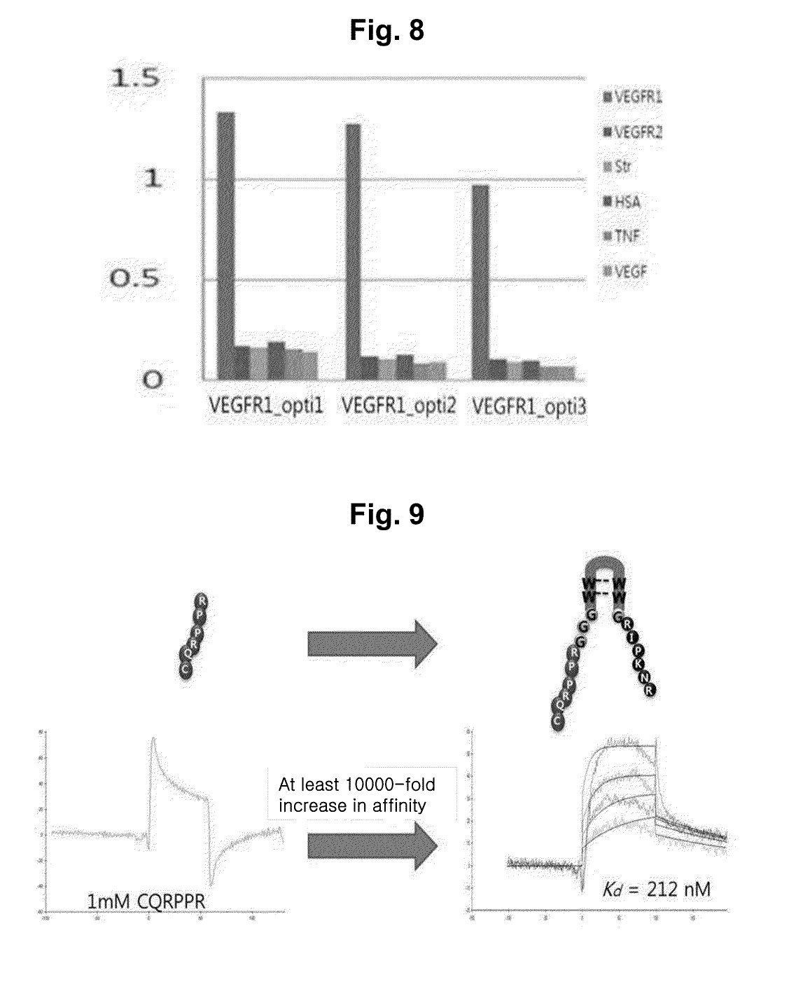

In order to investigate specificities of optimized peptides, ELISA was performed on VEGFR1, VEGFR2, streptavidin, HSA, TNF, and VEGF. It can be seen from FIG. 8 that the three peptides specifically bind to VEGFR1.

### Specificities of VEGFR1_Opti1, VEGFR1_Opti2, and VEGFR1_Opti3

Optimized peptide VEGFR1_opti1 and control peptide GNQWFI were synthesized, and affinities thereof were measured using the SPR Biacore system. As a result of affinity measurement, the affinity of VEGFR1_opti1 increased to 45 RU at 5 μM while control peptide WSHPQFEK increased 5 RU at 200 μM (FIG. 9). Through the results, it can be seen that the affinity increases about 360-fold. Therefore, it can be seen that the affinities to known peptides can be easily optimized several hundred-fold by the method suggested in the present invention.

### Example II

### Preparation of Affinity Optimization BPB

### Method

### Construction of BPB2_{EDB} Optimization Library

In order to construct a BPB2_{EDB} optimization library, two oligonucletides (5′-TTC TAT GCG GCC CAG CTG GCC (NNK)₆ GGA TCT TGG ACA TGG GAA AAC GGA AAA-3′; and 5′-AAC AGT TTC TGC GGC CGC TCC TCC TCC TTG CTC CAA CCT AAT AAT TCC CTT CCA TGT CCA TTT TCC GTT-3′) (BPB2_{EDB}_{_{—}}F1) and (5′-AAC AGT TTC TGC GGC CGC TCC TCC TCC TTG CTC CAA CCT AAT AAT TCC CTT CCA TGT CCA TTT TCC GTT-3′) (BPB2_{EDB}_{_{—}}B1), (N=A, T, G, or C); (K=G or T); (M=C or A) were synthesized. In order to prepare a double strand, BPB2_{EDB}_{_{—}}F1 4 μM (final concentration), BPB2_{EDB}_{_{—}}B1 4 μM (final concentration), 2.5 mM dNTP mix 4 μl, Ex TaqDNA polymerase 1 μl (10 U) (Takara, Seoul, Korea), and 10×PCR buffer 5 μl were mixed to prepare 25 mixture solutions having a total of 50 μl through the addition of distilled water. These mixture solutions were subjected to PCR (94° C. 5 min, 60 cycles: 30° C. 30 seconds, and 72° C. 30 seconds, and 72° C. 7 min), thereby preparing double strands, which were then purified using a PCR purification kit (GeneAll, Seoul, Korea). In order to ligate the BPB2_{EDB} insert gene to the pIGT2 phagemid vector (Ig therapy, Chuncheon, Korea), the insert gene and the pIGT2 phagemid vector were treated with restriction enzymes. About 11 μg of insert DNA was allowed to react with SfiI (NEB) and NofI (NEB) for 4 hrs, respectively, followed by purification using a PCR purification kit. Thereafter, about 40 μg of the pIGT2 phagemid vector was allowed to react with SfiI (NEB) and NofI (NEB) for 4 hrs, respectively, and then reacted with calf intestinal alkaline phosphatase (CIP; NEB, Ipswich) for 1 hr, followed by purification using a PCR purification kit. Then, 2.9 μg of the insert gene, which was obtained by quantifying the resultant products using a UV-visible light spectrophotometer (Ultrospec 2100 pro, Amersham Bioscience), was ligated to 12 μg of the pIGT2 phagemid vector at 18° C. for 15 hrs using T4 DNA ligase (Bioneer, Daejeon, Korea), followed by precipitation using ethanol, and then DNA was lysed with 100 μl of TE buffer. E. coli XL-1 competent cells were transformed with the resultant material by electroporation, finally constructing an 8×10⁷ library.

### Biopanning for Finding BPB2_{EDB}

2 ml of streptavidin (10 μg/ml) was added to 40 wells (50 μl per well) of a 96-well ELISA plate (Corning), and then allowed to stand at 4° C. overnight. The next day, only 20 wells were washed three times with 0.1% PBST (tween-20), and then biotin ED-B (10 μg/ml) was put thereinto, followed by standing at room temperature for 1 hr. Thereafter, all of the 40 wells were washed three times with 0.1% PBST, and blocked at room temperature for 2 hrs using 2% BSA diluted with PBS. Then, the solution was discarded, followed by washing three times with 0.1% PBST.

800 μl of a solution containing BPB2_{EDB} maturation recombinant phages and 200 μl of 10% BSA were added thereto, and in order to remove phages binding to streptavidin and BSA, the mixture was put into 20 wells coated with streptavidin and BSA, and allowed to stand at 27° C. for 1 hr. The supernatant was collected, and mixed with 100 μg/ml BPB1_{EDB}, followed by competitive biopanning. That is, in cases where this solution was transferred to 20 wells to which ED-B binds, followed by a reaction at 27° C. for 30 min, only phages having a more excellent affinity than BPB1_{EDB} can bind to EDB. The whole solution in the 20 wells was removed, and then the wells were washed ten times with 0.5% PBST. The phages were subsequently eluted for 20 min by adding 1 ml of 0.2 M glycine/HCl (pH 2.2) to each well (50 μl per well), and then 1 ml of the solution was collected in a tube, and neutralized by adding 150 μl of 2 M Tris-base (pH 9.0). In order to count input phages and elute phages for each biopanning, the phages were mixed with XL-1 BLUE cells (OD=0.7), and the mixture was plated on agar media containing ampicillin. In order to repeat panning, the phages were mixed with 10 ml of E. coli ER cells, and stirred cultured at a rate of 200 rpm for 1 hr at 37° C. After mixing with ampicillin (50 μg/ml) and 20 mM glucose, 2×10¹⁰ pfu of Ex helper phages were added, followed by stirring and culturing at a rate of 200 rpm for 1 hr at 37° C. After the culture liquid was centrifuged at 1,000×g for 10 min to remove the supernatant, the precipitated cells were resuspended in 40 ml LB liquid media containing ampicillin (50 μg/ml) and kanamycin (25 μg/ml), and then the solution was cultured at a rate of 200 rpm at 30° C. overnight. The culture liquid was centrifuged at 4,000×g for 20 min at 4° C. 8 ml of 5×PEG/NaCl [20% PEG(w/v) and 15% NaCl(w/v)] was added to the supernatant, and then the mixture was allowed to stand at 4° C. for 1 hr. After centrifugation, the PEG solution was completely removed, and the phage peptide pellets were lysed with 1 and PBS solution, which was then used in the second biopanning. Each panning step was carried out by the same method as described above except that the number of times of washing (0.5% PBST) increased 25 times and 35 times by steps.

### Construction and Screening BPB3_{EDB} and BPB4_{EDB} Optimization Libraries

In order to construct a BPB3_{EDB} optimization library, two oligonucleotides (5′-TTC TAT GCG GCC CAG CTG GCC NNK TGT GTT CTC CTA TTC AGG GAT CTT GGA CAT GGG AAA ACG GAA AA-3′) (BPB3_{EDB} F1) and (5′-AAC AGT TTC TGC GGC CGC TCC TCC TCC MNN TTG CTC CAA CCT AAT AAT TCC CTT CCA TGT CCA TTT TCC GTT-3′) (BPB3_{EDB}_{_{—}}B1), (N=A, T, G, or C); (K=G or T); (M=C or A) were synthesized.

In order to construct a BPB4_{EDB} optimization library, two oligonucleotides (5′-TTC TAT GCG GCC CAG CTG GCC NNK NNK TGT GTT CTC CTA TTC AGG GAT CTT GGA CAT GGG AAA ACG GAA AA-3′) (BPB4_{EDB} F1) and (5′-AAC AGT TTC TGC GGC CGC TCC TCC TCC MNN MNN TTG CTC CAA CCT AAT AAT TCC CTT CCA TGT CCA TTT TCC GTT-3′) (BPB4_{EDB}_{_{—}}B1), (N=A, T, G, or C); (K=G or T); (M=C or A) were synthesized.

In order to prepare a BPB3_{EDB} double strand, BPB3_{EDB}_{_{—}}F1 4 μM (final concentration), BPB3_{EDB}_{_{—}}B1 4 μM (final concentration), 2.5 mM dNTP mix 4 μl, Ex TaqDNA polymerase 1 μl (10 U) (Takara, Seoul, Korea), and 10×PCR buffer 5 μl were mixed to prepare 10 mixture solutions having a total of 50 μl through the addition of distilled water. Furthermore, in order to a BPB4_{EDB} double strand insert, BPB4_{EDB} F1 4 μM (final concentration), BPB4_{EDB}_{_{—}}B1 4 μM (final concentration), 2.5 mM dNTP mix 4 μl, Ex TaqDNA polymerase 1 μl (10 U) (Takara, Seoul, Korea), and 10×PCR buffer 5 μl to prepare 10 mixture solutions having a total of 50 μl through the addition of distilled water.

These mixture solutions were subjected to PCR (94° C. 5 min, 60 cycles: 30° C. 30 seconds, and 72° C. 30 seconds, and 72° C. 7 min), thereby preparing double strands, which were then purified using a PCR purification kit (GeneAll, Seoul, Korea). In order to ligate the BPB3_{EDB} and BPB4_{EDB} insert genes to the pIGT2 phagemid vectors (Ig therapy, Chuncheon, Korea), respectively, the insert genes and the pIGT2 phagemid vector were treated with restriction enzymes. About 10 μg of insert DNA was allowed to react with SfiI (NEB) and NofI (NEB) for 4 hrs, respectively, followed by purification using a PCR purification kit. Thereafter, about 40 μg of the pIGT2 phagemid vector was allowed to react with SfiI (NEB) and NofI (NEB) for 4 hrs, respectively, and then reacted with calf intestinal alkaline phosphatase (CIP; NEB, Ipswich) for 1 hr, followed by purification using a PCR purification kit. Then, 2.9 μg of the insert gene, which was obtained by quantifying the reaction product using a UV-visible light spectrophotometer (Ultrospec 2100 pro, Amersham Bioscience), was ligated to 12 μg of the pIGT2 phagemid vector at 18° C. for 15 hrs using T4 DNA ligase (Bioneer, Daejeon, Korea), followed by precipitation using ethanol, and then DNA was lysed with 100 μl of TE buffer. E. coli XL-1 competent cells were transformed with the resultant material by electroporation, finally constructing 1×10⁶ BPB3_{EDB} and BPB4_{EDB} libraries.

### Search of Phage Peptide Specific to ED-B Protein (Phage ELISA)

XL1-BLUE cells were infected with phages collected in the fourth biopanning step from the BPB2_{EDB}, BPB3_{EDB}, and BPB4_{EDB} optimization libraries, and then were plated such that the number of plaques per plate is 100-200. Thereafter, 60 plaques were inoculated in 2 ml and of LB-ampicillin (50 μg/ml) media using a sterile tip, and subjected to shaking culture at 37° C. for 5 hrs. 5×10⁹ pfu (OD=0.8-1) of Ex helper phages were added, followed by stirring and culturing at a rate of 200 rpm for 1 hr at 37° C. After the culture liquid was centrifuged at 1,000×g for 10 min to remove the supernatant, the precipitated cells were resuspended in 1 and LB liquid media containing ampicillin (50 μg/ml) and kanamycin (25 μg/ml), and then the solution was stirred and cultured at a rate of 200 rpm at 30° C. overnight. The culture liquid was centrifuged at 10,000×g for 20 min at 4° C. to collect the supernatant, and then 2% skim milk was added, which was used in the phage peptide research.

5 μg/ml streptavidin was put in 30 wells (50 μl per well) of a 96-well ELISA plate, and 10 μg/ml BSA was put in 30 wells (50 μl per well), followed by standing at 4° C. overnight. The next day, only 30 streptavidin wells were washed three times with 0.1% PBST (tween-20), and biotin ED-B (10 μg/ml) was put therein, followed by standing at room temperature for 1 hr.

All the wells were washed three times with 0.1% PBST, and blocked at room temperature for 2 hrs using 2% skim milk diluted with PBS. Then, the solution was discarded, followed by washing with 0.1% PBST three times. 100 μl of the phage peptide solution, amplified from each clone, was dispensed into the wells to which the ED-B protein adheres and the wells to which the BSA protein adheres, and allowed to stand at 27° C. for 1.5 hrs. After washing with 0.1% PBST solution 10 times, the HRP-conjugated anti-M13 antibody (GE Healthcare) was diluted to 1:1,000, which was then allowed to react at 27° C. for 1 hr. After washing with 0.1% PBST five times, 100 μl of the TMB solution was dispensed into each well to induce a colorimetric reaction, and then the reaction was stopped by adding 100 μl of 1 M HCl. The absorbance was measured at 450 nm to select phages in which the absorbance of ED-B was 20-fold higher than that of BSA. XL1 cells were infected with these phages, and the cells were plated such that the number of plaques per plate is 100-200. Thereafter, the plaques were inoculated in 4 ml of LB-ampicillin (50 μg/ml) culture liquid using a sterile tip, and subjected to shaking culture at 37° C. for one day. Plasmids were purified using a plasmid preparation kit, and then sequencing was commissioned. As a sequencing primer, 5′-GATTACGCCAAGCTTTGGAGC-3′, which is a vector sequence, was used.

### Binding Strength Assay (SPR)

The bipodal peptide binder found from BPB2_{EDB}, BPB3_{EDB}, and BPB4_{EDB} maturation libraries were synthesized (Anygen, Korea). The affinity measurement was conducted using BIAcore X (Biacore AB, Uppsala, Sweden). 2,000 RU of biotin-EDB was allowed to flow on the streptavidin SA chip (Biacore) for immobilization. PBS (pH 7.4) was used as a running buffer, and kinetics at different concentrations was measured under a flow rate of 30 μl per minute. The affinity was calculated using BlAevaluation software (Biacore AB, Uppsala, Sweden).

### Results

### Method for Constructing BPB Optimization Library

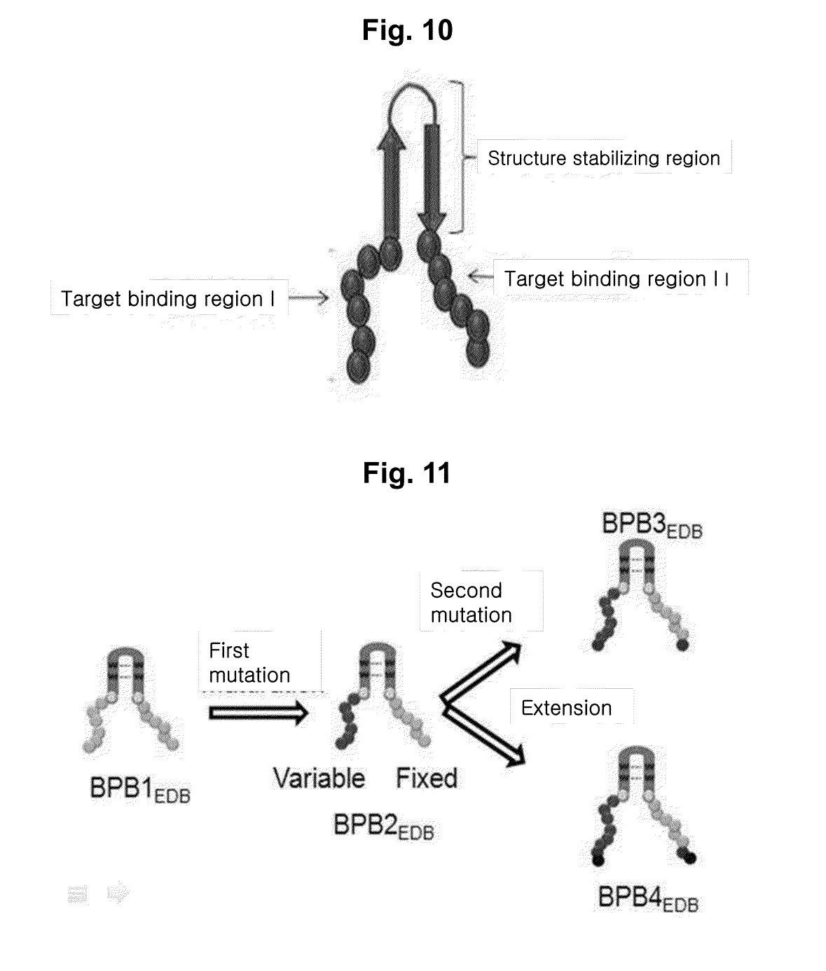

A stable β-hairpin motif was used as a bipodal peptide binder (BPB) scaffold. Especially, Trpzip, which stabilizes the structure of the β-hairpin motif by an interaction between tryptophan and tryptophan amino acids, was used. Each six amino acids were randomly arranged at the N-terminus and the C-terminus of Trpzip as a frame, respectively, thereby forming two variable regions (FIG. 10). A procedure of constructing an affinity optimization library of BPB is shown in FIG. 11. First, a library, in which one binding region of a bipodal-peptide binder (BPB1) found with respect to one target is fixed and the other binding region thereof is again randomized, was constructed, and the library was used for screening the target to find BPB2. Then, a library, in which each one or two random sequences are added to ends of both binding regions of BPB2, was constructed, and the library was used for screening to find BPB3 and BPB4, thereby optimizing BPB1.

### Construction and Screening of BPB2 Optimization Library

The BPB2 optimization library (XXXXXXGSWTWENGKWTWKGIIRLEQ, X=random amino acid) with a size of 8×10⁷ was constructed. Biopanning was performed on the ED-B protein three times using the BPB2 optimization library, and the ratio of output phage/input phage collected from each panning step was determined.

TABLE 1
Biopanning on ED-B domain

| Panning | Input phage | Elute phage | |
|---|---|---|---|
| round | (pfu) | (pfu) | yield |
| 1 | 4 × 10¹⁰ | 6.3 × 10⁷ | 1.6 × 10⁻³ |
| 2 | 1 × 10¹⁰ | 2.8 × 10⁸ | 28 × 10⁻³ |
| 3 | 2.5 × 10¹⁰ | 1.2 × 10⁹ | 50 × 10⁻³ |

Phage ELISA after First Affinity Optimization

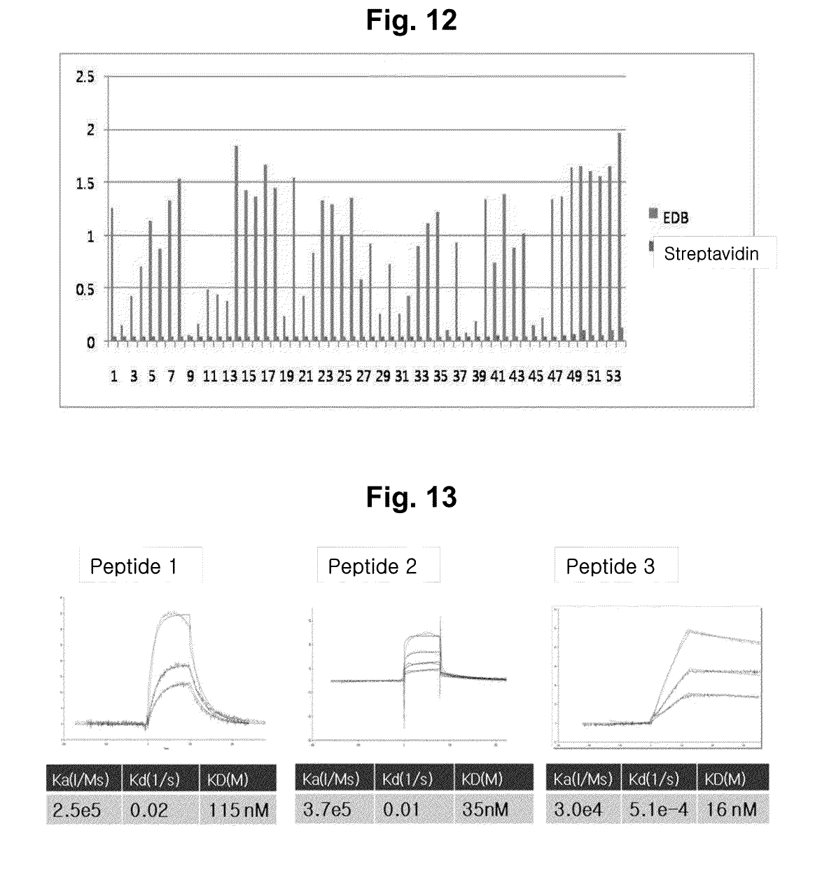

Phages collected from the last panning step of the BPB2 affinity optimization library were secured in a plaque form. After 54 phages were amplified from each plaque, ELISA was performed on ED-B and streptavidin (see FIG. 12). In most cases, the absorbance of ED-B was higher than that of streptavidin. DNA sequencing was performed on 24 clones in which the absorbance of ED-B was at least 20-fold higher than that of streptavidin. A total of three repeated peptide sequences were obtained:

### 1: WGGPVRGSWTWENGKWTWKGIIRLEQ (SEQ ID NO: 47);

### 2: ADGRVRGSWTWENGKWTWKGIIRLEQ (SEQ ID NO: 48);

### 3: CSSPIQGSWTWENGKWTWKGIIRLEQ (SEQ ID NO: 49).

### Affinity Measurement

Peptides 1, 2, and 3 were synthesized, and the affinities thereof were measured using the SPR Biacore system. As a result of measurement, the K_{D} value was 115 nM for peptide 1, 35 nM for peptide 2, and 16 nM for peptide 3 (see FIG. 13).

### Construction and Screening of Second Optimization Library

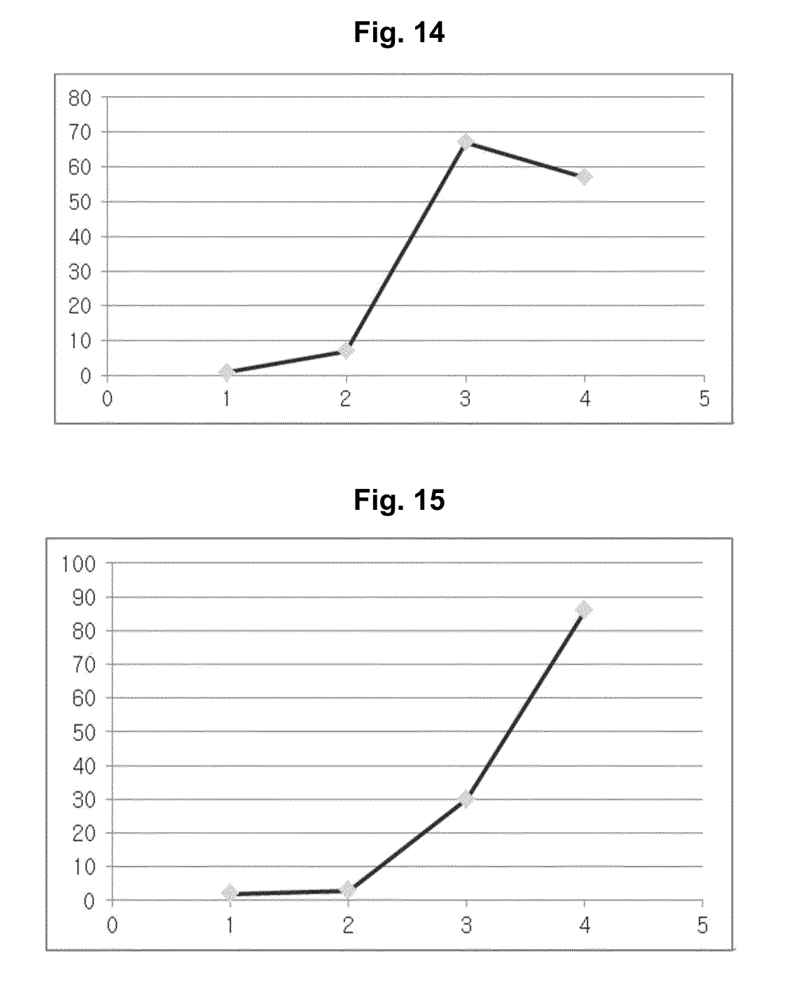

In order to again optimize peptide 3 (CSSPIQGSWTWENGKWTWKGIIRLEQ) showing the best affinity in the BPB2 peptides obtained after the first optimization, a library, in which each one or two variable regions were added to both ends, was constructed with a size of 10⁶. The BPB3 optimization library (XCSSPIQGSWTWENGKWTWKGIIRLEQX, X=random amino acid) and the BPB4 optimization library (XXCSSPIQGSWTWENGKWTWKGIIRLEQXX, X=random amino acid) were constructed with a size of 10⁶, respectively. Biopanning was performed on the ED-B protein four times using the libraries, and the ratios of output phage/input phage collected from each panning step were determined (FIGS. 14-15).

Phage ELISA after Second Optimization

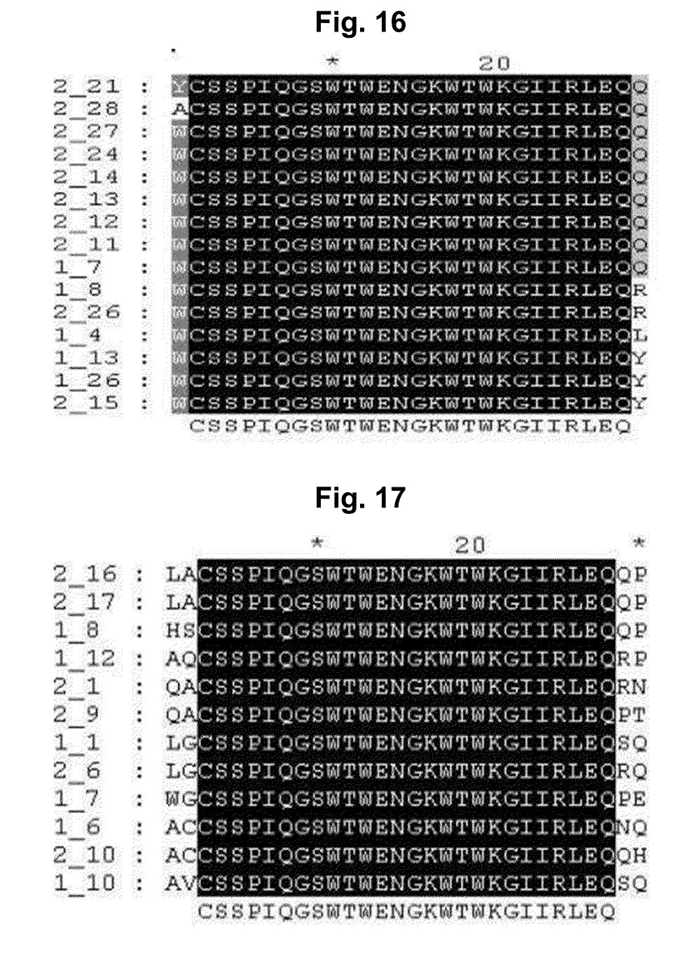

Phages collected from the last panning step of the BPB3 and BPB4 affinity optimization libraries were secured in a plaque form. After 60 phages were amplified from each plaque, ELISA was performed on ED-B and streptavidin, and DNA sequencing was performed on clones in which the absorbance of ED-B was at least 20-fold higher than that of streptavidin. Through this, the sequences shown in FIGS. 16 and 17 were obtained.

### Affinity Measurement

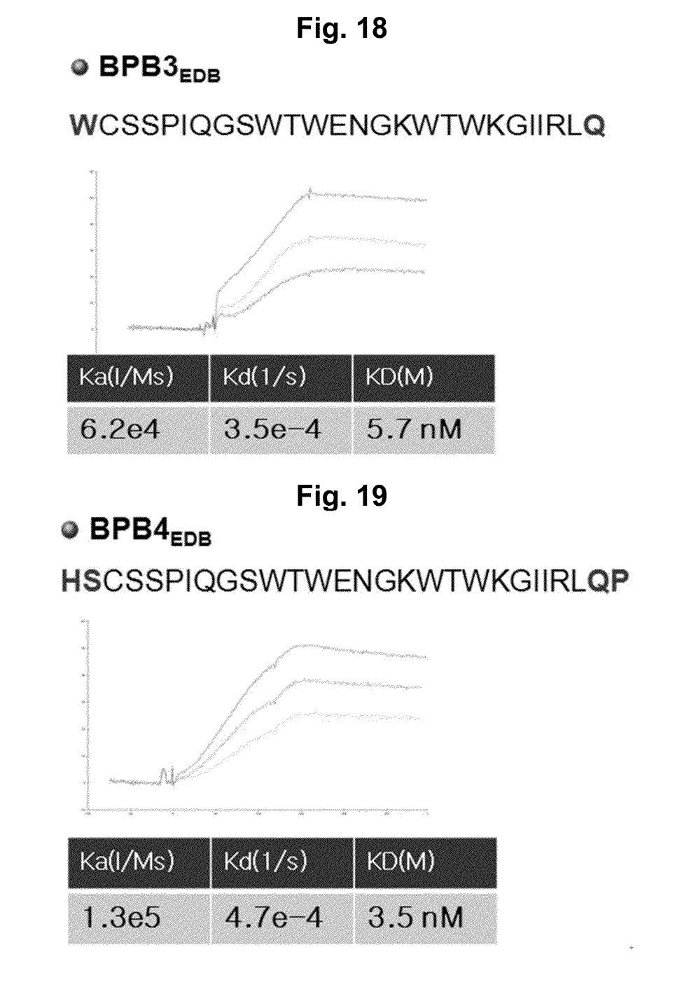

BPB3 and BPB4 peptides were synthesized, and affinities thereof were measured using a SPR Biacore system. As a result of the affinity measurement, BPB3 showed a K_{D} value of 5.7 nM and BPB4 showed a K_{D} value of 3.5 nM, which correspond to improved affinity values compared with BPB2 (FIGS. 18 and 19).

### Affinity Comparison

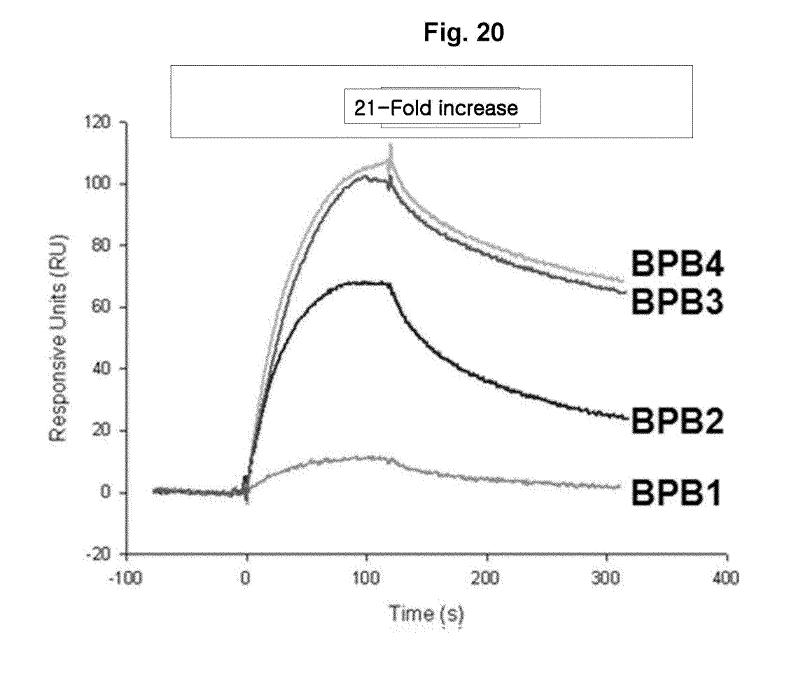

Affinities of 250 nM BPB1_{EDB}, BPB2_{EDB}, BPB3_{EDB}, and BPB4_{EDB} were compared with each other using an SPR Biacore system (FIG. 20). It can be seen from FIG. 20 that the affinity was improved through the optimization procedure. Finally, it was proved that the affinity of BPB4 increased 21-fold compared with BPB1 through the optimization system, and thus BPBs exhibiting a several nM-level affinity can be easily obtained (table 2).

TABLE 2
Target binding kinetics of BPB1, BPB2, BPB3, and BPB4 to
target binding

| | | | K_{D} [M] = |
|---|---|---|---|
| BPB_name | Kₒₙ [M⁻¹S⁻¹] | K_{off} [S⁻¹] | K_{off}/Kₒₙ |
| BPB1_{EDB} | 1.0 × 10⁴ | 8.0 × 10⁻⁴ | 75 × 10⁻⁹ |
| BPB2_{EDB} | 3.0 × 10⁴ | 5.1 × 10⁻⁴ | 16 × 10⁻⁹ |
| BPB3_{EDB} | 6.2 × 10⁴ | 3.6 × 10⁻⁴ | 5.7 × 10⁻⁹ |
| BPB4_{EDB} | 1.3 × 10⁵ | 4.7 × 10⁻⁴ | 3.5 × 10⁻⁹ |

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for increasing the target affinity of a peptide binding to a protein target, the method comprising:
(a) obtaining a known peptide having a binding affinity to the protein target;
(b) providing a library of KPI-bipodal peptide binder including; (i) a structure stabilizing region including parallel, antiparallel, or parallel and antiparallel amino acid strands with interstrand non-covalent bonds; and (ii) a known peptide, as target binding region I, binding to one terminus of the structure stabilizing region, and target binding region II including n amino acids on the other terminus of the structure stabilizing region;
(b) contacting the library with a target; and
(c) selecting a KPI-bipodal peptide binder binding to the target.

2. The method of claim 1, wherein the KPI-bipodal peptide binder selected from step (c) has a target affinity increasing 2- to 10000-fold compared with the known peptide.

3. The method of claim 1, wherein the KPI-bipodal peptide binder selected from step (c) has a dissociation constant (K_{D}) value of 0.1-10000 nM to the protein target.

4. A bipodal peptide binder (BPB) having a target affinity which is increased by the method of any one of claims 1 to 3.

5. A nucleic acid molecule coding the bipodal peptide binder of claim 4.


SEQUENCE LISTING (49 sequences)

SEQ ID NO: 1 - protein, 12 aa, Artificial Sequence
  Trpzip1
SWTWEGNKWTWK

SEQ ID NO: 2 - protein, 12 aa, Artificial Sequence
  Trpzip2
SWTWENGKWTWK

SEQ ID NO: 3 - protein, 12 aa, Artificial Sequence
  Trpzip3
SWTWEPNKWTWK

SEQ ID NO: 4 - protein, 16 aa, Artificial Sequence
  gb1,  41-56
GEWTYDDATKTFTVTE

SEQ ID NO: 5 - protein, 16 aa, Artificial Sequence
  Trpzip4
GEWTWDDATKTWTWTE

SEQ ID NO: 6 - protein, 16 aa, Artificial Sequence
  Trpzip5
GEWTYDDATKTFTWTE

SEQ ID NO: 7 - protein, 16 aa, Artificial Sequence
  Trpzip6
GEWTWDDATKTWTVTE

SEQ ID NO: 8 - protein, 16 aa, Artificial Sequence
  Trpzip7
GEWHWDDATKTWVWTE

SEQ ID NO: 9 - protein, 16 aa, Artificial Sequence
  Trpzip8
GEWVWDDATKTWHWTE

SEQ ID NO: 10 - protein, 16 aa, Artificial Sequence
  Trpzip9
GEWVWDDATKTWVWTE

SEQ ID NO: 11 - protein, 12 aa, Artificial Sequence
  (T3V)-HP6
KYVWSNGKWTVE

SEQ ID NO: 12 - protein, 12 aa, Artificial Sequence
  Espinosa-GB1(b)
RWQYVNGKFTVQ

SEQ ID NO: 13 - protein, 16 aa, Artificial Sequence
  GB1m2
GEWTYNPATGKFTVTE

SEQ ID NO: 14 - protein, 16 aa, Artificial Sequence
  GB1m3
KKWTYNPATGKFTVQE

SEQ ID NO: 15 - protein, 16 aa, Artificial Sequence
  HP5W4
KKWTYNPATGKWTWQE

SEQ ID NO: 16 - protein, 16 aa, Artificial Sequence
  HP5W
KKYTWNPATGKWTVQE

SEQ ID NO: 17 - protein, 16 aa, Artificial Sequence
  HP5A
KKYTWNPATGKATVQE

SEQ ID NO: 18 - protein, 12 aa, Artificial Sequence
  HP7
KTWNPATGKWTE

SEQ ID NO: 19 - protein, 10 aa, Artificial Sequence
  Chignolin
GYDPETGTWG

SEQ ID NO: 20 - protein, 26 aa, Artificial Sequence
  BPB-EB-B1
MSADKSGSWTWENGKWTWKGQVRTRD

SEQ ID NO: 21 - protein, 26 aa, Artificial Sequence
  BPB-ED-B2
HCSSAVGSWTWENGKWTWKGIIRLEQ

SEQ ID NO: 22 - protein, 26 aa, Artificial Sequence
  BPB-ED-B3
HSQGSPGSWTWENGKWTWKGRYSHRA

SEQ ID NO: 23 - protein, 26 aa, Artificial Sequence
  BPB-VEGF1
HANFFQGSWTWENGKWTWKGWKYNQS

SEQ ID NO: 24 - protein, 26 aa, Artificial Sequence
  BPB-VEGF2
ASPFWAGSWTWENGKWTWKGWVPSNA

SEQ ID NO: 25 - protein, 26 aa, Artificial Sequence
  BPB-VEGF3
HAFYYTGSWTWENGKWTWKGWPVTTS

SEQ ID NO: 26 - protein, 26 aa, Artificial Sequence
  BPB-VEGF4
YGAYPWGSWTWENGKWTWKGWRVSRD

SEQ ID NO: 27 - protein, 26 aa, Artificial Sequence
  BPB-VEGF5
AAPTSFGSWTWENGKWTWKGWQMWHR

SEQ ID NO: 28 - protein, 26 aa, Artificial Sequence
  BPB-VCAM1
QARDCTGSWTWENGKWTWKGPSICPI

SEQ ID NO: 29 - protein, 26 aa, Artificial Sequence
  BPB-nAchR1
EASFWLGSWTWENGKWTWKGKGTLNR

SEQ ID NO: 30 - protein, 26 aa, Artificial Sequence
  BPB-nAchR2
YAYPLLGSWTWENGKWTWKGWYQKWI

SEQ ID NO: 31 - protein, 26 aa, Artificial Sequence
  BPB-nAchR3
ASLPAWGSWTWENGKWTWKGWSTRTA

SEQ ID NO: 32 - protein, 26 aa, Artificial Sequence
  BPB-BSA1
AASPYKGSWTWENGKWTWKGGWRMKM

SEQ ID NO: 33 - protein, 26 aa, Artificial Sequence
  BPB-BSA2
SANSLYGSWTWENGKWTWKGTSRQRW

SEQ ID NO: 34 - protein, 26 aa, Artificial Sequence
  BPB-BSA3
YAHVYYGSWTWENGKWTWKGHRVTQT

SEQ ID NO: 35 - protein, 26 aa, Artificial Sequence
  BPB-BSA4
YGAYPWGSWTWENGKWTWKGWRVSRD

SEQ ID NO: 36 - protein, 26 aa, Artificial Sequence
  BPB-BSA5
YAHFGWGSWTWENGKWTWKGTTDSQS

SEQ ID NO: 37 - protein, 26 aa, Artificial Sequence
  BPB-MyD88-1
HSHAFYGSWTWENGKWTWKGNPGWWT

SEQ ID NO: 38 - protein, 26 aa, Artificial Sequence
  BPB-MyD88-2
ASTINFGSWTWENGKWTWKGYTRRWN

SEQ ID NO: 39 - protein, 35 aa, Artificial Sequence
  Leucine zipper for BPB
GGSMKQLEDKVEELLSKNYHLENEVARLKKLVGER

SEQ ID NO: 40 - protein, 41 aa, Artificial Sequence
  BPBLeu-ED-B-1
CSSPIQGGSMKQLEDKVEELLSKNYHLENEVARLKKLVGER

SEQ ID NO: 41 - protein, 41 aa, Artificial Sequence
  BPBLeu-ED-B-2
IIRLEQGGSMKQLEDKVEELLSKNYHLENEVARLKKLVGER

SEQ ID NO: 42 - protein, 30 aa, Artificial Sequence
  Strep_opti1
WSHPQFEKGGGSWTWENGKWTWKGAHPQVR

SEQ ID NO: 43 - protein, 30 aa, Artificial Sequence
  Strep_opti2
WSHPQFEKGGGSWTWENGKWTWKGTLIHPM

SEQ ID NO: 44 - protein, 28 aa, Artificial Sequence
  VEGFR1_opti1
GNQWFIGGGSWTWENGKWTWKGRIPKNR

SEQ ID NO: 45 - protein, 28 aa, Artificial Sequence
  VEGFR1_opti2
GNQWFIGGGSWTWENGKWTWKGKLQNPM

SEQ ID NO: 46 - protein, 28 aa, Artificial Sequence
  VEGFR1_opti3
GNQWFIGGGSWTWENGKWTWKGQQAIQP

SEQ ID NO: 47 - protein, 26 aa, Artificial Sequence
  BPB-EDB-1
WGGPVRGSWTWENGKWTWKGIIRLEQ

SEQ ID NO: 48 - protein, 26 aa, Artificial Sequence
  BPB-EDB-2
ADGRVRGSWTWENGKWTWKGIIRLEQ

SEQ ID NO: 49 - protein, 26 aa, Artificial Sequence
  BPB-EDB-3
CSSPIQGSWTWENGKWTWKGIIRLEQ